SEQUENCE LISTING (29 sequences)

SEQ ID NO: 1 - DNA, 1101 bp, Escherichia coli serotype O157H7 rfbE gene
atgaaaatgaaatatataccagtttaccaaccgtcattgacaggaaaagaaaaagaatatgtaaatgaatgtctggactcaacgtggatttcatcaaaaggaaactatattcagaagtttgaaaataaatttgcggaacaaaaccatgtgcaatatgcaactactgtaagtaatggaacggttgctcttcatttagctttgttagcgttaggtatatcggaaggagatgaagttattgttccaacactgacatatatagcatcagttaatgctataaaatacacaggagccacccccattttcgttgattcagataatgaaacttggcaaatgtctgttagtgacatagaacaaaaaatcactaataaaactaaagctattatgtgtgtccatttatacggacatccatgtgatatggaacaaattgtagaactggccaaaagtagaaatttgtttgtaattgaagattgcgctgaagcctttggttctaaatataaaggtaaatatgtgggaacatttggagatatttctacttttagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactaggaccgcagaggaaagagaggaattaaggaatcaccttgcagataaactcatcgaaacaaggccagttttttaccctgtccacacgatgccaatgtactcggaaaaatatcaaaagcaccctatagctgaggatcttggttggcgtggaattaatttacctagtttccccagcctatcgaatgagcaagttatttatatttgtgaatctattaacgaattttatagtgataaatag

SEQ ID NO: 2 - DNA, 334 bp, Escherichia coli serotype O157H7 E-co-B71
gatatttctacttttagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaa

SEQ ID NO: 3 - DNA, 332 bp, Escherichia coli serotype O157H7 E-co-B73
tagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactagga

SEQ ID NO: 4 - DNA, 325 bp, Escherichia coli serotype O157H7 E-co-B74
tttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactag

SEQ ID NO: 5 - DNA, 327 bp, Escherichia coli serotype O157H7 E-co-B75
tttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactagga

SEQ ID NO: 6 - DNA, 331 bp, Escherichia coli serotype O157H7 E-co-B76
tagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactagg

SEQ ID NO: 7 - DNA, 335 bp, Escherichia coli serotype O157H7 E-co-B81
agcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactaggaccgc

SEQ ID NO: 8 - DNA, 347 bp, Escherichia coli serotype O157H7 E-co-B8
atttctacttttagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactaggaccgc

SEQ ID NO: 9 - DNA, 330 bp, Escherichia coli serotype O157H7 E-co-B86
gcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactagga

SEQ ID NO: 10 - DNA, 344 bp, Escherichia coli serotype O157H7 E-co-B8
gatatttctacttttagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactag

SEQ ID NO: 11 - DNA, 332 bp, Escherichia coli serotype O157H7 E-co-B94
atttctacttttagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaat

SEQ ID NO: 12 - DNA, 353 bp, Escherichia coli serotype O157H7 E-co-B96
gagatatttctacttttagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttattggatggtctcaattctaactaggaccgca

SEQ ID NO: 13 - DNA, 320 bp, Escherichia coli serotype O157H7 E-co-B100
atatttctacttttagcttttttggaaataaaactattactacaggtgaaggtggaatggttgtcacgaatgacaaaacactttatgaccgttgtttacattttaaaggccaaggattagctgtacataggcaatattggcatgacgttataggctacaattataggatgacaaatatctgcgctgctataggattagcccagttagaacaagctgatgattttatatcacgaaaacgtgaaattgctgatatttataaaaaaaatatcaacagtcttgtacaagtccacaaggaaagtaaagatgtttttcacacttat

SEQ ID NO: 14 - DNA, 107 bp, Artificial Sequence
  Eschericia coli O157 consensus sequence
aggtggaatggttgtcacgaatgacaaaacactttatgaccgttgtttacattttaaaggccaaggattagctgtacataggcaatattggcatgacgttataggct

SEQ ID NO: 15 - DNA, 28 bp, Artificial Sequence
  Eschericia coli O157 exclusive sequence
accgttgtttacattttaaaggccaagg

SEQ ID NO: 16 - DNA, 18 bp, Artificial Sequence
  Forward primer sequence
aggtggaatggttgtcac

SEQ ID NO: 17 - DNA, 18 bp, Artificial Sequence
  Reverse primer sequence
agcctataacgtcatgcc

SEQ ID NO: 18 - DNA, 35 bp, Artificial Sequence
  Molecular Beacon #3 sequence
cgcaccgttgtttacattttaaaggccaaggtgcg

SEQ ID NO: 19 - DNA, 35 bp, Artificial Sequence
  Reverse complement Molecular Beacon #3 sequence
cgcaccttggcctttaaaatgtaaacaacggtgcg

SEQ ID NO: 20 - DNA, 28 bp, Artificial Sequence
  Reverse complement Molecular Beacon #3 loop sequence
ccttggcctttaaaatgtaaacaacggt

SEQ ID NO: 21 - DNA, 23 bp, Artificial Sequence
  Molecular beacon #1 and #2 loop sequence
ccgttgtttacattttaaaggcc

SEQ ID NO: 22 - DNA, 33 bp, Artificial Sequence
  Molecular beacon #1 sequence
cgcgcccgttgtttacattttaaaggccgcgcg

SEQ ID NO: 23 - DNA, 23 bp, Artificial Sequence
  Reverse complement Molecular beacon #1 and #2 loop sequence
ggcctttaaaatgtaaacaacgg

SEQ ID NO: 24 - DNA, 33 bp, Artificial Sequence
  Reverse complement Molecular beacon #1 sequence
cgcgcggcctttaaaatgtaaacaacgggcgcg

SEQ ID NO: 25 - DNA, 35 bp, Artificial Sequence
  Molecular beacon #2 sequence
cgagcgccgttgtttacattttaaaggcccgctcg

SEQ ID NO: 26 - DNA, 35 bp, Artificial Sequence
  Reverse complement Molecular beacon #2 sequence
cgagcgggcctttaaaatgtaaacaacggcgctcg

SEQ ID NO: 27 - DNA, 37 bp, Artificial Sequence
  Molecular beacon O157rfbE sequence
cgctatggtgaaggtggaatggttgtcacgaatagcg

SEQ ID NO: 28 - DNA, 32 bp, Artificial Sequence
  Forward primer O157BF sequence
aaatataaaggtaaatatgtgggaacatttgg

SEQ ID NO: 29 - DNA, 28 bp, Artificial Sequence
  Reverse primer O157BR sequence
tggcctttaaaatgtaaacaacggtcat

(19) United States
(12) Patent Application Publication
(10) Pub. No.: US 2008/0254449 A1
(43) Pub. Date: Oct. 16, 2008

(54) Polynucleotides For the Detection of Escherichia Coli 0157

(76) Inventors: Daniel Plante, Laval (CA); Eliane Ubalijoro, Les Cedres (CA); Alexandre Hebert, Laval (CA); Gregory Taylor, Ste-Therese (CA); Peggy Constant, Montreal (CA)

Correspondence Address: K&L Gates LLP, STATE STREET FINANCIAL CENTER, One Lincoln Street, BOSTON, MA, 02111-2950, US

(21) Appl. No.: 10/592,337

(22) PCT Filed: Nov. 30, 2004
(86) PCT No.: PCT/CA2004/002059

§ 371 Date: Jun. 12, 2008

Related U.S. Application Data
(60) Provisional application No. 60/552,151, filed on Mar. 11, 2004.

(51) Int. Cl. C12Q 1/68; C07H 21/00; C12P 19/34

(52) U.S. Cl. 435/6; 536/24.32; 536/24.33; 435/91.1

(57) ABSTRACT

Polynucleotide primers and probes for the amplification and detection of E. coli O157 in a test sample are provided. The primers and probes can be used in real time diagnostic assays for rapid detection of E. coli O157 in a variety of situations, including clinical samples, microbiological pure cultures, food, and environmental and pharmaceutical quality control processes. Kits comprising the primers and probes are also provided.

### FIELD OF THE INVENTION

The present invention pertains to the field of detection of microbial contaminants and in-particular to the detection of contamination by Escherichia coli O157.

### BACKGROUND OF THE INVENTION

Escherichia coli O157 strains are responsible for a large number of reported cases of food poisoning throughout the world. This bacterium is commonly associated with contamination of foods such as ground beef, milk, milk products, alfalfa sprouts, lettuce, fruit juices and cured meats. Within 24 to 96 hours of ingestion, individuals infected by the pathogen may develop symptoms such as stomach cramps, abdominal pain, bloody diarrhoea, and, in more severe cases, haemolytic uremic syndrome, in which red blood cells are destroyed and the kidneys fail. In order to prevent E. coli O157 infections, methods of detection can be utilized that identify the presence of the bacteria in food, prior to consumer availability and consumption. However, due to relatively quick rates of food spoilage, many detection techniques, which require long time periods, are not time and cost effective. For example, a number of detection technologies require the culturing of bacterial samples for time periods of up to eight days. However, in that time, the product being tested must be placed in circulation for purchase and consumption.

In addition, E. coli O157 contamination of foods can be difficult to detect as low levels of E. coli can be swamped by high numbers of other bacteria. The infective dose of E. coli O157 is estimated to be between 10 to 100 organisms only making detection of low levels of this pathogen important. Current detection methods for E. coli O157 are based on enrichment in selective broth and subsequent isolation on selective agar or on immunomagnetic bead separation followed by culture and identification on selective medium. Both of these methods are time-consuming and labour intensive. ELISA-based methods of detection are also available, as are immuno-blotting methods, but these techniques may have limited sensitivity.

Other methods have been described in the art for the detection of bacterial contaminants such as E. coli, including PCR-based assays and assays based on nucleic acid hybridization.

For example, International Patent Application PCT/AU98/00315 (WO 98/50531) and U.S. Patent Application 2003/0018349 describe nucleic acid molecules derived from bacterial genes (including E.coli O157:H7 genes) encoding transferases or enzymes for the transport or processing of a polysaccharide unit of the bacterial capsule and methods of detecting bacteria in samples using these nucleic acid molecules. A PCR-based method for detecting E. coli O157 has been described by Desmarchelier et al. (J. Clin. Microbiol. (1998) 36:1801-1804) that involves amplification of a region of the O-antigen synthesis genes followed by gel electrophoresis and Southern blot analysis to confirm the identify of the amplified fragment. The method was capable of identifying two serotypes of E. coli O157; the O157:H7 and O157:H—serotypes. A similar PCR-based protocol based on the amplification of the rfbB region of the O-antigen synthesis genes is described by Maurer et al. (Appl. Environ. Microbiol. (1999) 65:2954-2960). Although such PCR-based methods of detection are more rapid than traditional methods requiring the culture of bacterial samples, they are still relatively time consuming and subject to post-PCR contamination during the running of the agarose gel.

Methods of specifically detecting the E. coli O157 serotype O157:H7 have also been described. For example, U.S. Pat. No. 5,654,417 describes a DNA fragment that is useful for specific detection of the serotype E. coli O157:H7 in food and faecal samples and nucleic acid probes comprising at least 15 nucleotides that are capable of hybridising to this DNA fragment. U.S. Pat. No. 6,365,723 and U.S. Patent Application 2003/0023075 describe genomic sequences that are present in the serotype E.coli O157:H7 but absent from E. coli K12 and isolated polynucleotides comprising at least 25 nucleotides of one of these sequences that can be used as diagnostic probes. These methods and probes relate to the detection of the O157:H7 serotype only and not to the detection of E. coli O157 in general.

A useful modification of PCR- and hybridisation technologies provides for the concurrent amplification and detection of the target sequence (i.e. in “real time”) through the use of specially adapted oligonucleotide probes. Examples of such probes include molecular beacon probes (Tyagi et al., (1996) Nature Biotechnol. 14:303-308), TaqMan® probes (U.S. Pat. Nos. 5,691,146 and 5,876,930) and Scorpion probes (Whitcombe et al., (1999) Nature Biotechnol. 17:804-807).

A molecular beacon probe designed to specifically detect the E. coli O157:H7 serotype has been described (Fortin et al., (2001) Analytical Biochem. 289:281-288). The probe was designed to hybridise to an amplified target sequence from the rfbE O-antigen synthesis gene of E.coli O157:H7 that is either 496 base pair (bp) or 146 bp in length, depending on the primers used. The probe was also able to detect E. coli O157:NM and O157:H—serotypes. The PCR required a 4-step PCR protocol in order to obtain good sensitivity, however, the use of primers that yielded the shorter amplified region (146 bp) resulted in poor sensitivity with either the 4-step or 3-step PCR protocol.

The enzymes involved in the production of the O side chain of the lipopolysaccharide (LPS) of E. coli O157 are encoded by genes in the rfb operon (Bilge, et al. (1996) Infection and Immunity 64(11):4795-801; Maurer, et al. (1999) Appl. Environ. Microbiol. 65(7):2954-2960; Stevens, et al. (1970) J. American Chem. Soc. 92(10):3160-31688; Stroeher, et al. (1995) Gene 166(1):33-42; Stroeher, et al. (1992) PNAS (USA) 89(7):2566-2570; Tarr, et al. (2000) J. Bacteriol. 182(21):6183-6191). The RfbE protein (encoded by the rfbE gene of this operon) is a perosamine synthetase involved in the production of 4-amino-4,6-dideoxy-d-mannose (perosamine) from GDP-4-keto-6-dideoxymannose. Perosamine is a component of the O polysaccharide side chain, which constitutes the outermost part of the LPS molecule.

Another detection method based on the E.coli rfbE gene is described in International Patent Application PCT/AT02/00222 (WO 03/010332). This application describes a test kit designed to amplify, trap and detect enterohaemorrhagic E. coli strains. The methodology employs a specifically modified amplification and detection assay that involves trapping of the amplicon by a “trapping probe” bound to a solid surface and subsequent detection of the trapped amplicon by a second probe that specifically binds to the amplicon.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide polynucleotides for the detection of Escherichia coli O157. In accordance with one aspect of the present invention, there is provided a combination of polynucleotides for the amplification and detection of a portion of an E. coli O157 rfbE gene, said portion being less than about 475 nucleotides in length and comprising at least 65 consecutive nucleotides of the sequence set forth in SEQ ID NO:14, said combination of polynucleotides comprising: (a) a first polynucleotide primer comprising at least 7 consecutive nucleotides of the sequence as set forth in SEQ ID NO:1; (b) a second polynucleotide primer comprising at least 7 consecutive nucleotides of a sequence complementary to SEQ ID NO:1; and (c) a polynucleotide probe comprising at least 7 consecutive nucleotides of the sequence as set forth in SEQ ID NO:14, or the complement thereof.

In accordance with another aspect of the invention, there is provided a pair of polynucleotide primers for amplification of a portion of an E. coli O157 rfbE gene, said portion being less than about 475 nucleotides in length and comprising at least 65 consecutive nucleotides of the sequence set forth in SEQ ID NO:14, said pair of polynucleotide primers comprising: (a) a first polynucleotide primer comprising at least 7 consecutive nucleotides of the sequence as set forth in SEQ ID NO:1; and (b) a second polynucleotide primer comprising at least 7 consecutive nucleotides of a sequence complementary to SEQ ID NO:1.

In accordance with another aspect of the invention, there is provided a method of detecting E. coli O157 in a sample, said method comprising: (a) providing a test sample suspected of containing, or known to contain, E. coli O157 nucleic acids; and (b) contacting said test sample with a combination of polynucleotides of the invention under conditions that permit amplification and detection of a portion of an E. coli O157 rfbE gene, wherein detection of said a portion of the E. coli O157 rfbE gene indicates the presence E. coli O157 in the sample.

In accordance with another aspect of the invention, there is provided a kit for the detection of an E. coli O157 rfbE target sequence, said target sequence being less than about 475 nucleotides in length and comprising at least 65 consecutive nucleotides of the sequence set forth in SEQ ID NO:14, said kit comprising: (a) a first polynucleotide primer comprising at least 7 consecutive nucleotides of the sequence as set forth in SEQ ID NO:1; (b) a second polynucleotide primer comprising at least 7 consecutive nucleotides of a sequence complementary to SEQ ID NO:1; and (c) a polynucleotide probe comprising at least 7 consecutive nucleotides of the sequence as set forth in SEQ ID NO:14, or the complement thereof.

In accordance with another aspect of the invention, there is provided an isolated E. coli O157 specific polynucleotide having the sequence as set forth in SEQ ID NO:14, or the complement thereof.

In accordance with another aspect of the invention, there is provided a polynucleotide primer of between 7 and 100 nucleotides in length for the amplification of a portion of an E. coli O157 rfbE gene, said polynucleotide primer comprising at least 7 consecutive nucleotides of the sequence as set forth in SEQ ID NO:14, or the complement thereof, with the proviso that the primer is other than SEQ ID NO:29.

In accordance with another aspect of the invention, there is provided a polynucleotide probe of between 7 and 100 nucleotides in length for detection of E. coli O157 nucleic acids, said polynucleotide probe comprising at least 7 consecutive nucleotides of the sequence as set forth in SEQ ID NO:14, or the complement thereof, with the proviso that the probe is other than SEQ ED NO:27.

In accordance with another aspect of the invention, there is provided a method of detecting E. coli O157 nucleic acids in a sample, said method comprising: (a) contacting a test sample suspected of containing, or known to contain, E. coli O157 nucleic acids with a polynucleotide probe of the invention under conditions that permit hybridisation of said probe to said E. coli O157 nucleic acids to form a probe:target hybrid, and (b) detecting any probe:target hybrid, wherein detection of said probe:target hybrid is indicative of the presence of said E. coli O157 nucleic acids in said sample.

In accordance with another aspect of the invention, there is provided a method of amplifying an E. coli O157 target nucleic acid sequence, said method comprising: (a) forming a reaction mixture comprising a test sample suspected of containing, or known to contain, an E. coli O157 target nucleic acid sequence, amplification reagents, and a pair of polynucleotide primers of the invention; and (b) subjecting the mixture to amplification conditions to generate at least one copy of said target nucleic acid sequence.

### BRIEF DESCRIPTION OF THE DRAWINGS

These and other features of the invention will become more apparent in the following detailed description in which reference is made to the appended drawings wherein:

FIG. 1 presents a multiple alignment showing conserved regions of a portion of the rfbE gene from E. coli O157 and related E. coli strains [SEQ ID NOs:2-13]. Shaded blocks highlight the following regions: bases 53 to 70 represent forward primer SEQ ID NO:16; bases 91 to 118 represent the binding site for molecular beacon #3[SEQ ID NO:18]; bases 142 to 159 represent reverse primer SEQ ID NO:17;

FIG. 2 presents the arrangement in one embodiment of the invention of PCR primers and a molecular beacon probe on the rfbE gene sequence. Numbers in parentheses indicate the positions of the first and last nucleotides of each feature on the PCR product generated with primers SEQ ID NOs:16 & 17;

FIG. 3 presents the secondary structure of a molecular beacon probe in accordance with one embodiment of the invention [SEQ ID NO:18]; and

FIG. 4 presents (A) the sequence of an E. coli O157 rfbE gene [SEQ ID NO:1]; (B) the sequence of a conserved region of the E. coli O157 rfbE gene [SEQ ID NO:14], which is unique to E. coli O157 isolates and (C) a 28 nucleotide sequence [SEQ ID NO:15] found within the conserved region, which is exclusive to E. coli O157 isolates.

### DETAILED DESCRIPTION OF THE INVENTION

The present invention is based on the identification of a highly conserved region (consensus sequence) that is common to strains of E. coli O157. The consensus sequence constitutes a suitable target sequence for the design of primers and probes capable of specifically amplifying and detecting E. coli O157 in a test sample. The consensus sequence provided by the present invention allows for the design of primers and probes that can amplify and detect various E. coli O157 serotypes. In one embodiment, the primers and probes are capable of amplifying and detecting three or more E. coli O157 serotypes. In another embodiment, the primers and probes are capable of amplifying and detecting four or more E. coli O157 serotypes. In a further embodiment, the primers and probes are capable of amplifying and detecting more than four E. coli O157 serotypes.

The present invention provides for primer and probe sequences capable of amplifying and/or detecting all or part of the consensus sequence that are suitable for use in detecting the presence of E. coli O157 bacteria in a range of samples including, but not limited to, clinical samples, microbiological pure cultures, food, and environmental and pharmaceutical quality control processes. In one embodiment, the invention provides diagnostic assays that can be carried out in real time and addresses the need for rapid detection of E. coli O157 in a variety of biological samples.

### Definitions

Unless defined otherwise, all technical and scientific terms used herein have the same meaning as commonly understood by one of ordinary skill in the art to which this invention belongs.

The term “polynucleotide,” as used herein, refers to a polymer of greater than one nucleotide in length of ribonucleic acid (RNA), deoxyribonucleic acid (DNA), hybrid RNA/DNA, modified RNA or DNA, or RNA or DNA mimetics. The polynucleotides may be single- or double-stranded. The term includes polynucleotides composed of naturally-occurring nucleobases, sugars and covalent internucleoside (backbone) linkages as well as polynucleotides having non-naturally-occurring portions which function similarly. Such modified or substituted polynucleotides are well-known in the art and for the purposes of the present invention, are referred to as “analogues.”

The terms “primer” and “polynucleotide primer,” as used herein, refer to a short, single-stranded polynucleotide capable of hybridizing to a complementary sequence in a nucleic acid sample. A primer serves as an initiation point for template-dependent nucleic acid synthesis. Nucleotides are added to a primer by a nucleic acid polymerase, which adds such nucleotides in accordance with the sequence of the template nucleic acid strand. A “primer pair” or “primer set” refers to a set of primers including a 5′ upstream primer that hybridizes with the 5′ end of the sequence to be amplified and a 3′ downstream primer that hybridizes with the complementary 3′ end of the sequence to be amplified. The term “forward primer,” as used herein, refers to a primer which anneals to the 5′ end of the sequence to be amplified. The term “reverse primer,” as used herein, refers to a primer which anneals to the complementary 3′ end of the sequence to be amplified.

The terms “probe” and “polynucleotide probe,” as used herein, refer to a polynucleotide used for detecting the presence of a specific nucleotide sequence (or “target nucleotide sequence”) in a sample. Probes specifically hybridize to a target nucleotide sequence, or the complementary sequence thereof, and may be single- or double-stranded.

The term “specifically hybridize,” as used herein, refers to the ability of a polynucleotide to bind detectably and specifically to a target nucleotide sequence. Polynucleotides specifically hybridize to target nucleotide sequences under hybridization and wash conditions that minimize appreciable amounts of detectable binding to non-specific nucleic acids. High stringency conditions can be used to achieve specific hybridization conditions as is known in the art. Typically, hybridization and washing are performed at high stringency according to conventional hybridization procedures and employing one or more washing step in a solution comprising 1-3×SSC, 0.1-1% SDS at 50-70° C. for 5-30 minutes.

The term “corresponding to” refers to a polynucleotide sequence that is identical to all or a portion of a reference polynucleotide sequence. In contradistinction, the term “complementary to” is used herein to indicate that the a polynucleotide sequence is identical to all or a portion of the complementary strand of a reference polynucleotide sequence. For illustration, the nucleotide sequence “TATAC” corresponds to a reference sequence “TATAC” and is complementary to a reference sequence “GTATA.”

The terms “hairpin” or “hairpin loop” refer to a single strand of DNA or RNA, the ends of which comprise complementary sequences, whereby the ends anneal together to form a “stem” and the region between the ends is not annealed and forms a “loop.” Some probes, such as molecular beacons, have such “hairpin” structure when not hybridized to a target sequence. The loop is a single-stranded structure containing sequences complementary to the target sequence, whereas the stem self-hybridises to form a double-stranded region and is typically unrelated to the target sequence. Nucleotides that are both complementary to the target sequence and that can self-hybridise can be included in the stem region.

The terms “target sequence” or “target nucleotide sequence,” as used herein, refer to a particular nucleic acid sequence in a test sample to which a primer and/or probe is intended to specifically hybridize. A “target sequence” is typically longer than the primer or probe sequence and thus can contain multiple “primer target sequences” and “probe target sequences.” A target sequence may be single or double stranded. The term “primer target sequence” as used herein refers to a nucleic acid sequence in a test sample to which a primer is intended to specifically hybridize. The term “probe target sequence” refers to a nucleic acid sequence in a test sample to which a probe is intended to specifically hybridize.

As used herein, the term “about” refers to a ±10% variation from the nominal value. It is to be understood that such a variation is always included in any given value provided herein, whether or not it is specifically referred to.

### Target Sequence

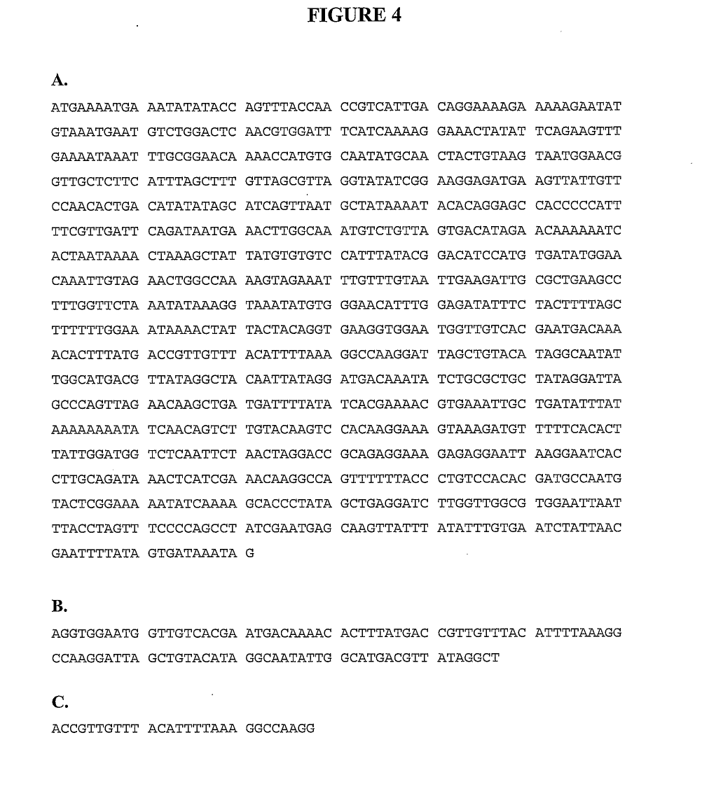

In order to identify highly conserved regions of the rfbE gene that could potentially serve as target sequences for specific probes, the rfbE gene sequences (having a general sequence corresponding to SEQ ID NO:1) from various E. coli O157 serotypes were subjected to a multiple alignment analysis. A 107 nucleotide region of the rfbE gene sequence, having a sequence corresponding to SEQ ID NO:14 (shown in FIG. 4B), was identified as being generally conserved in E. coli O157 isolates. This sequence is referred to herein as a consensus sequence.

Accordingly, the present invention provides an isolated E. coli O157 specific polynucleotide consisting of the consensus sequence as set forth in SEQ ID NO:14 (and shown in FIG. 4B), or the complement thereof, that can be used as a target sequence for the design of probes for the specific detection of E. coli O157.

It will be recognised by those skilled in the art that all, or a portion, of the consensus sequence set forth in SEQ ID NO:14 can be used as a target sequence for the specific detection of E. coli O157. Thus, in one embodiment of the invention, a target sequence suitable for the specific detection of E. coli O157 comprising at least 60% of the sequence set forth in SEQ ID NO:14, or the complement thereof, is provided. In another embodiment, the target sequence comprises at least 75% of the sequence set forth in SEQ ID NO:14, or the complement thereof. In a further embodiment, the target sequence comprises at least 80% of the sequence set forth in SEQ ID NO:14, or the complement thereof. Target sequences comprising at least 85%, 90%, 95% and 98% of the sequence set forth in SEQ ID NO:14, or the complement thereof, are also contemplated.

Alternatively, such portions of the consensus sequence can be expressed in terms of consecutive nucleotides of the sequence set forth in SEQ ID NO:14. Accordingly, target sequences comprising portions of the consensus sequence including at least 65, at least 70, at least 75, at least 80, at least 85, at least 90, at least 95, at least 100 and at least 105 consecutive nucleotides of the sequence set forth in SEQ ID NO:14, or the complement thereof, are contemplated. By “at least 65 consecutive nucleotides” it is meant that the target sequence may comprise any number of consecutive nucleotides between 65 and 107 of the sequence set forth in SEQ ID NO:14, thus this range includes portions of the consensus sequence that comprise at least 66, at least 67, at least 68, at least 69, etc, consecutive nucleotides of the sequence set forth in SEQ ID NO:14, or the complement thereof.

Within the 107 nucleotide consensus sequence, an additional highly conserved 28 nucleotide region, having a sequence corresponding to SEQ ID NO:15, was identified (shown in FIG. 4C). Accordingly, one embodiment of the present invention provides for target sequences that comprise a sequence corresponding to SEQ ID NO:15, or the complement thereof.

It will also be appreciated that the target sequence may include additional nucleotide sequences that are found upstream and/or downstream of the consensus sequence in the E. coli O157 genome. As the assays provided by the present invention typically include an amplification step, it may be desirable to select an overall length for the target sequence such that the assay can be conducted fairly rapidly. Thus, the target sequence typically has an overall length of less than about 475 nucleotides. In one embodiment, the target sequence has an overall length of less than about 450 nucleotides. In another embodiment, the target sequence has an overall length of less than about 425 nucleotides. In a further embodiment, the target sequence has an overall length of less than about 400 nucleotides. In other embodiments, the target sequence has an overall length of less than about 375, less than about 350, less than about 300, less than about 250, less than about 200, less than about 150 nucleotides and less than about 145 nucleotides. In a further embodiment, the target sequence has an overall length corresponding approximately to the length of the consensus sequence, i.e. about 107 nucleotides.

### Polynucleotide Primers and Probes

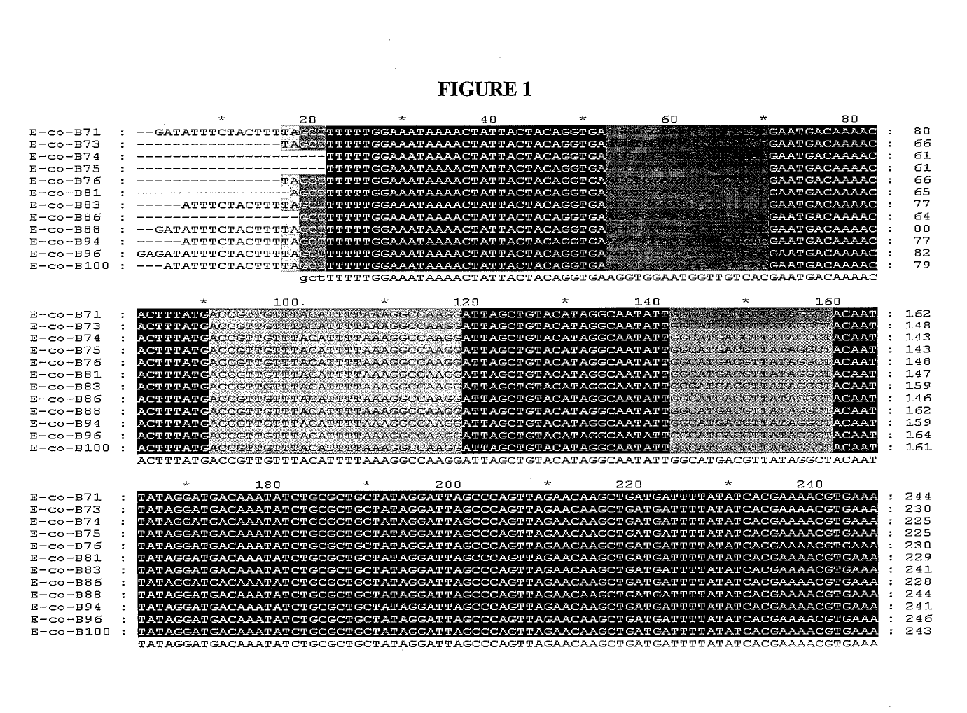

The present invention provides for polynucleotides for the amplification and/or detection of E. coli O157 nucleic acids in a sample. The polynucleotides of the invention comprise a sequence that corresponds to or is complementary to a portion of the E. coli O157 rfbE gene sequence and are capable of specifically hybridizing to E. coli O157 nucleic acids. In one embodiment, the polynucleotides of the invention comprise a sequence that corresponds to or is complementary to a portion of the E. coli O157 rfbE gene sequence as set forth in SEQ ID NO:1 (and shown in FIG. 4A). In a further embodiment, the polynucleotides of the invention comprise a sequence that corresponds to or is complementary to a portion of any one of the regions of the E. coli O157 rfbE gene sequence as set forth in SEQ ID NOs:2-13 (and shown in FIG. 1).

The polynucleotides of the present invention are generally between about 7 and about 100 nucleotides in length. One skilled in the art will understand that the optimal length for a selected polynucleotide will vary depending on its intended application (i.e. primer, probe or combined primer/probe) and on whether any additional features, such as tags, self-complementary “stems” and labels (as described below), are to be incorporated. In one embodiment of the present invention, the polynucleotides are between about 10 and about 100 nucleotides in length. In another embodiment, the polynucleotides are between about 12 and about 100 nucleotides in length. In other embodiments, the polynucleotides are between about 12 and about 50 nucleotides and between about 12 and about 35 nucleotides in length.

One skilled in the art will also understand that the entire length of the polynucleotide primer or probe does not need to correspond to or be complementary to the E. coli O157 rfbE gene sequence in order to specifically hybridize thereto. Thus, the polynucleotide primers and probes may comprise nucleotides at the 5′ and/or 3′ termini that are not complementary to the E. coli O157 rfbE gene sequence. Such non-complementary nucleotides may provide additional functionality to the primer/probe, for example, they may provide a restriction enzyme recognition sequence or a “tag” that facilitates detection, isolation or purification. Alternatively, the additional nucleotides may provide a self-complementary sequence that allows the primer/probe to adopt a hairpin configuration. Such configurations are necessary for certain probes, for example, molecular beacon and Scorpion probes.

The present invention also contemplates that one or more position within the polynucleotide can be degenerate, i.e. can be filled by one of two or more alternate nucleotides. As is known in the art, certain positions in a gene can vary in the nucleotide that is present at that position depending on the strain of bacteria that the gene originated from. Degenerate primers or probes are typically prepared by synthesising a “pool” of polynucleotide primers or probes that contains approximately equal amounts of polynucleotides containing the appropriate nucleotide at the degenerate position. By way of example, a polynucleotide having a degenerate position that could be filled by either an “A” or a “G” would be prepared by synthesizing a pool of polynucleotides containing approximately equal amounts of a polynucleotide having an A at the degenerate position and a polynucleotide containing a G at the degenerate position.

Typically, the polynucleotide primers and probes of the invention comprise a sequence of at least 7 consecutive nucleotides that correspond to or are complementary to a portion of the E. coli O157 rfbE gene sequence. As is known in the art, the optimal length of the sequence corresponding or complementary to the E. coli O157 rfbE gene sequence will be dependent on the specific application for the polynucleotide, for example, whether it is to be used as a primer or a probe and, if the latter, the type of probe. Optimal lengths can be readily determined by the skilled artisan.

In one embodiment, the polynucleotides comprise at least 10 consecutive nucleotides corresponding or complementary to a portion of the E. coli O157 rfbE gene sequence. In another embodiment, the polynucleotides comprise at least 12 consecutive nucleotides corresponding or complementary to a portion of the E. coli O157 rfbE gene sequence. In a further embodiment, the polynucleotides comprise at least 15 consecutive nucleotides corresponding or complementary to a portion of the E. coli O157 rfbE gene sequence. Other embodiments provide for polynucleotides comprising at least 16, at least 18, at least 20, at least 22, at least 24, at least 26, at least 27 and at least 28 consecutive nucleotides corresponding or complementary to a portion of the E. coli O157 rfbE gene sequence.

Sequences of exemplary polynucleotides of the invention are set forth in Table 1. Further non-limiting examples for the polynucleotides of the invention include polynucleotides that comprise at least 7 consecutive nucleotides of any one of SEQ ID NOs:14, 15, 16, 17, 20, 21 or 23.

TABLE 1
Exemplary polynucleotides of the invention

| | SEQ |
|---|---|
| | ID |
| Nucleotide sequence | NO |
| 5′-AGGTGGAATGGTTGTCAC-3′ | 16 |
| 5′-AGCCTATAACGTCATGCC-3′ | 17 |
| 5′-ACCGTTGTTTACATTTTAAAGGCCAAGG-3′ | 15 |
| 5′-CCTTGGCCTTTAAAATGTAAACAACGGT-3′ | 20 |
| 5′-CCGTTGTTTACATTTTAAAGGCC-3′ | 21 |
| 5′-GGCCTTTAAAATGTAAACAACGG-3′ | 23 |

### Primers

As indicated above, the polynucleotide primers of the present invention comprise a sequence that corresponds to or is complementary to a portion of the E. coli O157 rfbE gene sequence. In accordance with the invention, the primers are capable of amplifying a target nucleotide sequence comprising all or a portion of the 107 nucleotide consensus sequence as shown in SEQ ID NO:14. Accordingly, the present invention provides for primer pairs capable of amplifying an E. coli O157 target nucleotide sequence, wherein the target sequence is less than about 475 nucleotides in length and comprises at least 65 consecutive nucleotides of SEQ ID NO:14, or the complement thereof, as described above.

Thus, pairs of primers can be selected to comprise a forward primer corresponding to a portion of the E. coli O157 rfbE gene sequence upstream of or within the region of the gene corresponding to SEQ ID NO:14 and a reverse primer that it is complementary to a portion of the E. coli O157 rfbE gene sequence downstream of or within the region of the gene corresponding to SEQ ID NO:14. In accordance with one embodiment of the present invention, the primers comprise at least 7 consecutive nucleotides of the sequence set forth in SEQ ID NO:1. In another embodiment, the primers comprise at least 7 consecutive nucleotides of any one of SEQ ID NOs:2-13. In another embodiment, the primers comprise at least 7 consecutive nucleotides of the sequence set forth in SEQ ID NO:14.

As indicated above the polynucleotide primers of the present invention are between about 7 and about 100 nucleotides in length. In one embodiment, the primers are between about 10 and about 50 nucleotides in length. In another embodiment, the polynucleotides are between about 10 and about 40 nucleotides in length. In other embodiments, the polynucleotides are between about 10 and about 30 nucleotides and between about 10 and about 25 nucleotides in length.

Appropriate primer pairs can be readily determined by a worker skilled in the art. In general, primers are selected that specifically hybridize to a portion of the E. coli O157 rfbE gene sequence without exhibiting significant hybridization to non-E. coli O157 rfbE nucleic acids. In addition, the primers are selected to contain minimal sequence repeats and such that they show a low potential for dimer formation, cross dimer formation, hairpin structure formation and cross priming. Such properties can be determined by methods known in the art, for example, using the computer modelling program OLIGO® Primer Analysis Software (distributed by National Biosciences, Inc., Plymouth, Minn.).

Non-limiting examples of suitable primer sequences include SEQ ID NOs:16 and 17 shown in Table 1, as well as primers comprising at least 7 consecutive nucleotides of any one of SEQ ID NOs:14, 15, 16, 17, 20, 21 or 23.

### Probes

In order to specifically detect E. coli O157, the probe polynucleotides of the invention are designed to correspond to or be complementary to a portion of the consensus sequence shown in SEQ ID NO:14. The probe polynucleotides, therefore, comprise at least 7 consecutive nucleotides of the sequence set forth in SEQ ID NO:14, or the complement thereof. As indicated above, a highly conserved 28 nucleotide region was identified within the O157 consensus sequence. In one embodiment, therefore, the present invention provides for probe polynucleotides comprising at least 7 consecutive nucleotides of the sequence set forth in SEQ ID NO:15, or the complement thereof.

Non-limiting examples of suitable probe sequences include SEQ ID NOs:15, 20, 21 and 23 shown in Table 1, as well as probes comprising at least 7 consecutive nucleotides of any one of SEQ ID NOs:14, 15, 16, 17 or 21, or the complement thereof. In one embodiment of the present invention, the polynucleotide probes comprise at least 7 consecutive nucleotides of any one of SEQ ID NOs:15, 17 or 21, or the complement thereof.

Various types of probes known in the art are contemplated by the present invention. For example, the probe may be a hybridization probe, the binding of which to a target nucleotide sequence can be detected using a general DNA binding dye such as ethidium bromide, SYBR® Green, SYBR® Gold and the like. Alternatively, the probe can incorporate one or more detectable labels. Detectable labels are molecules or moieties a property or characteristic of which can be detected directly or indirectly and are chosen such that the ability of the probe to hybridize with its target sequence is not affected. Methods of labelling nucleic acid sequences are well-known in the art (see, for example, Ausubel et al., (1997 & updates) Current Protocols in Molecular Biology, Wiley & Sons, New York).

Labels suitable for use with the probes of the present invention include those that can be directly detected, such as radioisotopes, fluorophores, chemiluminophores, enzymes, colloidal particles, fluorescent microparticles, and the like. One skilled in the art will understand that directly detectable labels may require additional components, such as substrates, triggering reagents, light, and the like to enable detection of the label. The present invention also contemplates the use of labels that are detected indirectly. Indirectly detectable labels are typically specific binding members used in conjunction with a “conjugate” that is attached or coupled to a directly detectable label. Coupling chemistries for synthesising such conjugates are well-known in the art and are designed such that the specific binding property of the specific binding member and the detectable property of the label remain intact. As used herein, “specific binding member” and “conjugate” refer to the two members of a binding pair, i.e. two different molecules, where the specific binding member binds specifically to the probe, and the “conjugate” specifically binds to the specific binding member. Binding between the two members of the pair is typically chemical or physical in nature. Examples of such binding pairs include, but are not limited to, antigens and antibodies; avidin/streptavidin and biotin; haptens and antibodies specific for haptens; complementary nucleotide sequences; enzyme cofactors/substrates and enzymes; and the like.

In one embodiment of the present invention, the probe is labelled with a fluorophore. The probe may additionally incorporate a quencher for the fluorophore. Fluorescently labelled probes can be particularly useful for the real-time detection of target nucleotide sequences in a test sample. Examples of probes that are labelled with both a fluorophore and a quencher that are contemplated by the present invention include, but are not limited to, molecular beacon probes and TaqMan® probes. Such probes are well known in the art (see for example, U.S. Pat. Nos. 6,150,097; 5,925,517 and 6,103,476; Marras et al., “Genotyping single nucleotide polymorphisms with molecular beacons.” In Kwok, P. Y. (ed.), “Single nucleotide polymorphisms: methods and protocols,” Vol. 212, pp. 111-128, Humana Press, Totowa, N.J.)

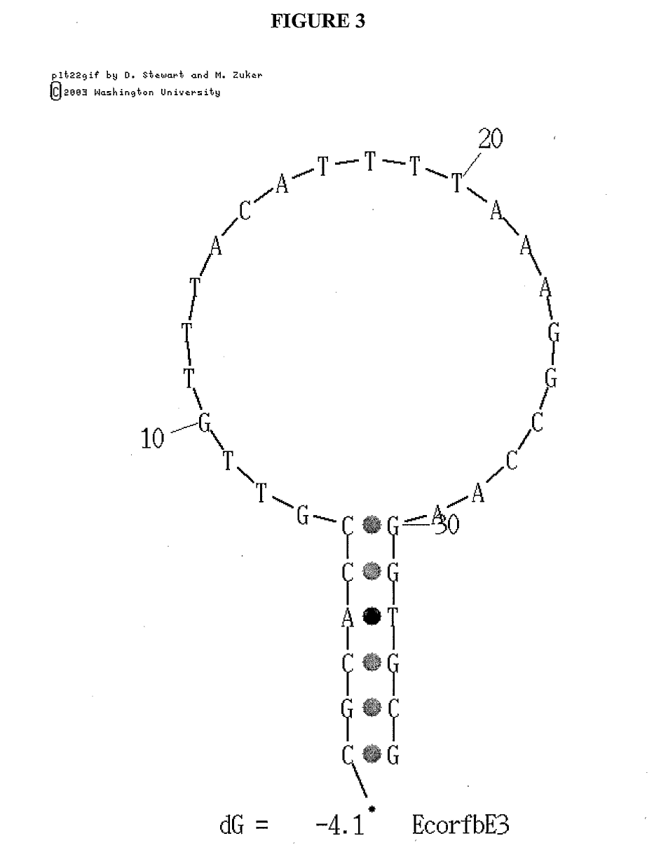

A molecular beacon probe is a hairpin shaped oligonucleotide sequence, which undergoes a conformational change when it hybridizes to a perfectly complementary target sequence. The secondary structure of a typical molecular beacon probe includes a loop sequence, which is capable of hybridizing to a target sequence and a pair of arm (or “stem”) sequences. One arm is attached to a fluorophore, while the other arm is attached to a quencher. The arm sequences are complementary to each other so as to enable the arms to hybridize together to form a molecular duplex and the beacon adopts a hairpin conformation in which the fluorophore and quencher are in close proximity and interact such that emission of fluorescence is prevented. Hybridization between the loop sequence and the target sequence forces the molecular beacon probe to undergo a conformational change in which arm sequences are forced apart and the fluorophore is physically separated from the quencher. As a result, the fluorescence of the fluorophore is restored. The fluorescence generated can be monitored and related to the presence of the target nucleotide sequence. If no target sequence is present in the sample, no fluorescence will be observed. This methodology, as described further below, can also be used to quantify the amount of target nucleotide in a sample. By way of example, FIG. 3 depicts the secondary structure of an exemplary hairpin loop molecular beacon (molecular beacon #3) having a sequence corresponding to SEQ ID NO:18.

Wavelength-shifting molecular beacon probes which incorporate two fluorophores, a “harvester fluorophore and an “emitter” fluorophore (see, Kramer, et al., (2000) Nature Biotechnology, 18:1191-1196) are also contemplated. When a wavelength-shifting molecular beacon binds to its target sequence and the hairpin opens, the energy absorbed by the harvester fluorophore is transferred by fluorescence resonance energy transfer (FRET) to the emitter, which then fluoresces. Wavelength-shifting molecular beacons are particularly suited to multiplex assays.

TaqMan® probes are dual-labelled fluorogenic nucleic acid probes that function on the same principles as molecular beacons. TaqMan® probes are composed of a polynucleotide that is complementary to a target sequence and is labelled at the 5′ terminus with a fluorophore and at the 3′ terminus with a quencher. TaqMan® probes, like molecular beacons, are typically used as real-time probes in amplification reactions. In the free probe, the close proximity of the fluorophore and the quencher ensures that the fluorophore is internally quenched. During the extension phase of the amplification reaction, the probe is cleaved by the 5′ nuclease activity of the polymerase and the fluorophore is released. The released fluorophore can then fluoresce and produce a detectable signal.

Linear probes comprising a fluorophore and a high efficiency dark quencher, such as the Black Hole Quenchers (BHQ™; Biosearch Technologies, Inc., Novato, Calif.) are also contemplated. As is known in the art, the high quenching efficiency and lack of native fluorescence of the BHQ™ dyes allows “random-coil” quenching to occur in linear probes labelled at one terminus with a fluorophore and at the other with a BHQ™ dye thus ensuring that the fluorophore does not fluoresce when the probe is in solution. Upon binding its target sequence, the probe stretches out spatially separating the fluorophore and quencher and allowing the fluorophore to fluoresce. One skilled in the art will appreciate that the BHQ™ dyes can also be used as the quencher moiety in molecular beacon or TaqMan® probes.

As an alternative to including a fluorophore and a quencher in a single molecule, two fluorescently labelled probes that anneal to adjacent regions of the target sequence can be used. One of these probes, a donor probe, is labelled at the 3′ end with a donor fluorophore, such as fluorescein, and the other probe, the acceptor probe, is labelled at the 5′ end with an acceptor fluorophore, such as LC Red 640 or LC Red 705. When the donor fluorophore is stimulated by the excitation source, energy is transferred to the acceptor fluorophore by FRET resulting in the emission of a fluorescent signal.

In addition to providing primers and probes as separate molecules, the present invention also contemplates polynucleotides that are capable of functioning as both primer and probe in an amplification reaction. Such combined primer/probe polynucleotides are known in the art and include, but are not limited to, Scorpion probes, duplex Scorpion probes, Lux™ primers and Amplifluor™ primers.

Scorpion probes consist of, from the 5′ to 3′ end, (i) a fluorophore, (ii) a specific probe sequence that is complementary to a portion of the target sequence and is held in a hairpin configuration by complementary stem loop sequences, (iii) a quencher, (iv) a PCR blocker (such as, hexethylene glycol) and (v) a primer sequence. After extension of the primer sequence in an amplification reaction, the probe folds back on itself so that the specific probe sequence can bind to its complement within the same DNA strand. This opens up the hairpin and the fluorophore can fluoresce. Duplex Scorpion probes are a modification of Scorpion probes in which the fluorophore-coupled probe/primer containing the PCR blocker and the quencher-coupled sequence are provided as separate complementary polynucleotides. When the two polynucleotides are hybridized as a duplex molecule, the fluorophore is quenched. Upon dissociation of the duplex when the primer/probe binds the target sequence, the fluorophore and quencher become spatially separated and the fluorophore fluoresces. The Amplifluor Universal Detection System also employs fluorophore/quencher combinations and is commercially available from Chemicon International (Temecula, Calif.).

In contrast, Lux™ primers incorporate only a fluorophore and adopt a hairpin structure in solution that allows them to self-quench. Opening of the hairpin upon binding to a target sequence allows the fluorophore to fluoresce.

Suitable fluorophores and/or quenchers for use with the polynucleotides of the present invention are known in the art (see for example, Tyagi et al., Nature Biotechnol., 16:49-53 (1998); Marras et al., Genet. Anal.: Biomolec. Eng., 14:151-156 (1999)). Many fluorophores and quenchers are available commercially, for example from Molecular Probes (Eugene, Oreg.) or Biosearch Technologies, Inc. (Novato, Calif.). Examples of fluorophores that can be used in the present invention include, but are not limited to, fluorescein and fluorescein derivatives, such as 6-carboxyfluoroscein (FAM), 5′-tetrachlorofluorescein phosphoroamidite (TET), tetrachloro-6-carboxyfluoroscein, VIC and JOE, 5-(2′-aminoethyl)aminonaphthalene-1-sulphonic acid (EDANS), coumarin and coumarin derivatives, Lucifer yellow, Texas red, tetramethylrhodamine, 5-carboxyrhodamine, cyanine dyes (such as Cy5) and the like. Pairs of fluorophores suitable for use as FRET pairs include, but are not limited to, fluorescein/rhodamine, fluorescein/Cy5, fluorescein/Cy5.5, fluorescein/LC Red 640, fluorescein/LC Red 750, and phycoerythrin/Cy7. Quenchers include, but are not limited to, 4′-(4-dimethylaminophenylazo)benzoic acid (DABCYL), 4-dimethylaminophenylazophenyl-4′-(DABMI), tetramethylrhodamine, carboxytetramethylrhodamine (TAMRA), BHQ™ dyes and the like.

Methods of selecting appropriate sequences for and preparing the various primers and probes are known in the art. For example, the polynucleotides can be prepared using conventional solid-phase synthesis using commercially available equipment, such as that available from Applied Biosystems USA Inc. (Foster City, Calif.), DuPont, (Wilmington, Del.), or Milligen (Bedford, Mass.). Methods of coupling fluorophores and quenchers to nucleic acids are also in the art.

In one embodiment of the present invention, the probe polynucleotide is a molecular beacon. In general, in order to form a hairpin structure effectively, molecular beacons are at least 17 nucleotides in length. In accordance with this aspect of the invention, therefore, the molecular beacon probe is typically between about 17 and about 40 nucleotides in length. Within the probe, the loop sequence that corresponds to or is complementary to the target sequence typically is about 7 to about 32 nucleotides in length, while the stem (or “arm”) sequences are each between about 4 and about 9 nucleotides in length. As indicated above, part of the stem sequences of a molecular beacon may also be complementary to the target sequence. In one embodiment of the present invention, the loop sequence of the molecular beacon is between about 10 and about 30 nucleotides in length. In other embodiments, the loop sequence of the molecular beacon is between about 15 and about 30 nucleotides in length.

In accordance with the present invention, the loop region of the molecular beacon probe comprises at least 7 consecutive nucleotides of the sequence as set forth in SEQ ID NO:14, or the complement thereof. In a specific embodiment, the loop region of the molecular beacon probe comprises at least 7 consecutive nucleotides of the sequence as set forth in SEQ ID NO:15, or the complement thereof.

Amplification and/or Detection

The present invention provides for methods of detecting E. coli O157 serotypes in a sample by contacting a sample known to contain or suspected of containing an E. coli O157 target nucleotide sequence with one or more of the polynucleotide probes described above under conditions that permit hybridisation of the probe(s) to the target nucleotide sequence. The hybridised probe(s) can then be detected by conventional methods. In an alternative embodiment, the present invention provides for methods of detecting E. coli O157 by the amplifying the target nucleotide sequence prior to detection. Amplification of the target nucleotide sequence prior to detection allows for the screening of test samples containing only small amounts of these sequences.

Accordingly, in one embodiment of the present invention, E. Coli O157 detection involves subjecting a test sample to an amplification reaction in order to obtain an amplification product, or amplicon comprising the target sequence, and detecting the target sequence.

As used herein, an “amplification reaction” refers to a process that increases the number of copies of a particular nucleic acid sequence by enzymatic means. Amplification procedures are well-known in the art and include, but are not limited to, polymerase chain reaction (PCR), TMA, rolling circle amplification, nucleic acid sequence based amplification (NASBA), strand displacement amplification (SDA) and Q-beta replicase amplification. One skilled in the art will understand that for use in certain amplification techniques the primers described above may need to be modified, for example, SDA primers comprise additional nucleotides near the 5′ end that constitute a recognition site for a restriction endonuclease. Similarly, NASBA primers comprise additional nucleotides near the 5′ end that are not complementary to the target sequence but which constitute an RNA polymerase promoter. Polynucleotides thus modified are considered to be within the scope of the present invention.

In one embodiment of the present invention, the target sequence is amplified by PCR. PCR is a method known in the art for amplifying a nucleotide sequence using a heat stable polymerase and a pair of primers, one primer (the forward primer) complementary to the (+)-strand at one end of the sequence to be amplified and the other primer (the reverse primer) complementary to the (−)-strand at the other end of the sequence to be amplified. Newly synthesized DNA strands can subsequently serve as templates for the same primer sequences and successive rounds of strand denaturation, primer annealing, and strand elongation, produce rapid and highly specific amplification of the target sequence. PCR can thus be used to detect the existence of a defined sequence in a DNA sample. The term “PCR” as used herein refers to the various forms of PCR known in the art including, but not limited to, quantitative PCR, reverse-transcriptase PCR, real-time PCR, hot start PCR, long PCR, LAPCR, multiplex PCR, touchdown PCR, and the like. “Real-time PCR” refers to a PCR reaction in which the amplification of a target sequence is monitored in real time by, for example, the detection of fluorescence emitted by the binding of a labelled probe to the amplified target sequence.

In one embodiment, the present invention thus provides for amplification of a portion of an E. coli O157 rfbE gene of less than about 475 nucleotides in length and comprising at least 65 consecutive nucleotides of the sequence set forth in SED ID NO:14 using pairs of polynucleotide primers, each member of the primer pair comprising at least 7 consecutive nucleotides of the sequence as set forth in SEQ ID NO:1, or the complement thereof.

The product of the amplification reaction can be detected by a number of means known to individuals skilled in the art. Examples of such detection means include, for example, gel electrophoresis and/or the use of polynucleotide probes. In one embodiment of the invention, the amplification products are detected through the use of polynucleotide probes. Such polynucleotide probes are described in detail above.

A further embodiment of the invention, therefore, provides for amplification and detection of a portion of an E. coli O157 rfbE gene of less than about 475 nucleotides in length and comprising at least 65 consecutive nucleotides of the sequence set forth in SED ID NO:14 using a combination of polynucleotides, the combination comprising one or more polynucleotide primers comprising at least 7 consecutive nucleotides of the sequence as set forth in SEQ ID NO:1, or the complement thereof, and a polynucleotide probe comprising at least 7 consecutive nucleotides of the sequence as set forth in SEQ ID NO:14, or the complement thereof.

It will be readily appreciated that a procedure that allows both amplification and detection of target E. coli O157 nucleic acid sequences to take place concurrently in a single unopened reaction vessel would be advantageous. Such a procedure would avoid the risk of “carry-over” contamination in the post-amplification processing steps, and would also facilitate high-throughput screening or assays and the adaptation of the procedure to automation. Furthermore, this type of procedure allows “real time” monitoring of the amplification reaction, as discussed above, as well as more conventional “end-point” monitoring. In one embodiment, the detection is accomplished in real time in order to facilitate rapid detection. In a specific embodiment, detection is accomplished in real time through the use of a molecular beacon probe.

The present invention thus provides for methods to specifically amplify and detect E. coli O157 nucleic acid sequences in a test sample in a single tube format using the polynucleotide primers, and optionally one or more probes, described herein. Such methods may employ dyes, such as SYBR® Green or SYBR® Gold that bind to the amplified target sequence, or an antibody that specifically detects the amplified target sequence. The dye or antibody is included in the reaction vessel and detects the amplified sequences as it is formed. Alternatively, a labelled polynucleotide probe (such as a molecular beacon or TaqMan® probe) distinct from the primer sequences, which is complementary to a region of the amplified sequence, may be included in the reaction, or one of the primers may act as a combined primer/probe, such as a Scorpion probe. Such options are discussed in detail above.

Thus, a general method of detecting E. coli O157 in a sample is provided that comprises contacting a test sample suspected of containing, or known to contain, an E.coli O157 target nucleotide sequence with a combination of polynucleotides comprising at least one polynucleotide primer and at least one polynucleotide probe or primer/probe, as described above, under conditions that permit amplification and detection of said target sequence, and detecting any amplified target sequence as an indication of the presence of E. coli O157 in the sample. A “test sample” as used herein is a biological sample suspected of containing, or known to contain, an E. coli O157 target nucleotide sequence.

In one embodiment of the present invention, a method using the polynucleotide primers and probes or primer/probes is provided to specifically amplify and detect an E.coli O157 target nucleotide sequence in a test sample, the method generally comprising the steps of:
(a) forming a reaction mixture comprising a test sample, amplification reagents, at least one labelled polynucleotide probe sequence capable of specifically hybridising to a portion of an E.coli O157 target nucleotide sequence and at least one polynucleotide primer corresponding to or complementary to a portion of an E.coli O157 rfbE gene comprising said target nucleotide sequence;
(b) subjecting the mixture to amplification conditions to generate at least one copy of the target nucleotide sequence, or a nucleic acid sequence complementary to the target nucleotide sequence;
(c) hybridizing the probe to the target nucleotide sequence or the nucleic acid sequence complementary to the target sequence, so as to form a probe:target hybrid; and
(d) detecting the probe:target hybrid as an indication of the presence of the E. coli O157 target nucleotide sequence in the test sample.

The term “amplification reagents” includes conventional reagents employed in amplification reactions and includes, but is not limited to, one or more enzymes having nucleic acid polymerase activity, enzyme cofactors (such as magnesium or nicotinamide adenine dinucleotide (NAD)), salts, buffers, nucleotides such as deoxynucleotide triphosphates (dNTPs; for example, deoxyadenosine triphosphate, deoxyguanosine triphosphate, deoxycytidine triphosphate and deoxythymidine triphosphate) and other reagents that modulate the activity of the polymerase enzyme or the specificity of the primers.

It will be readily understood by one skilled in the art that step (b) of the above method can be repeated several times prior to step (c) by thermal cycling the reaction mixture by techniques known in the art and that steps (b), (c) and (d) may take place concurrently such that the detection of the amplified sequence takes place in real time. In addition, variations of the above method can be made depending on the intended application of the method, for example, the polynucleotide probe may be a combined primer/probe, or it may be a separate polynucleotide probe, in which case two different polynucleotide primers are used. Additional steps may be incorporated before, between or after those listed above as necessary, for example, the test sample may undergo enrichment, extraction and/or purification steps to isolate nucleic acids therefrom prior to the amplification reaction, and/or the amplified product may be submitted to purification/isolation steps or further amplification prior to detection, and/or the results from the detection step (d) may be analysed in order to quantify the amount of target present in the sample or to compare the results with those from other samples. These and other variations will be apparent to one skilled in the art and are considered to be within the scope of the present invention.

In one embodiment of the present invention, the method is a real-time PCR assay utilising two polynucleotide primers and a molecular beacon probe. In another embodiment, the target sequence is a portion of an E. coli O157 rfbE gene of less than about 475 nucleotides in length and comprising at least 65 consecutive nucleotides of the sequence set forth in SED ID NO:14, the polynucleotide probe comprises at least 7 consecutive nucleotides of the sequence as set forth in SEQ ID NO:14, or the complement thereof, and the polynucleotide primers comprise at least 7 consecutive nucleotides of the sequence as set forth in SEQ ID NO:1, or the complement thereof.

Diagnostic Assays to Detect E. coli O157

The present invention provides for diagnostic assays using the polynucleotide primers and/or probes that can be used for highly specific detection of E. coli O157 in a test sample. The diagnostic assays comprise amplification and detection of E. coli O157 nucleic acids as described above. The diagnostic assays can be qualitative or quantitative and can involve real time monitoring of the amplification reaction or more conventional end-point monitoring. The diagnostic assays of the present invention can be used to detect a variety of E. coli O157 serotypes. In one embodiment, the diagnostic assays are capable of detecting three or more E. coli O157 serotypes. In another embodiment, the diagnostic assays are capable of detecting four or more E. coli O157 serotypes. In a further embodiment, the diagnostic assays are capable of detecting more than four E. coli O157 serotypes.

In one embodiment, the invention provides for diagnostic assays that do not require post-amplification manipulations and minimise the amount of time required to conduct the assay. For example, in a specific embodiment, there is provided a diagnostic assay, utilising the primers and probes described herein, that can be completed using real time PCR technology in, at most, 54 hours and generally 24 hours or less.

Such diagnostic assays are particularly useful in the detection of E. coli O157 contamination of various foodstuffs. Thus, in one embodiment, the present invention provides a rapid and sensitive diagnostic assay for the detection of E. coli O157 contamination of a food sample. Foods that can be analysed using the diagnostic assays include, but are not limited to, dairy products such as milk, including raw milk, cheese, yoghurt, ice cream and cream; raw, cooked and cured meats and meat products, such as beef, pork, lamb, mutton, poultry (including turkey, chicken), game (including rabbit, grouse, pheasant, duck), minced and ground meat (including ground beef, ground turkey, ground chicken, ground pork); eggs; fruits and vegetables; nuts and nut products, such as nut butters; seafood products including fish and shellfish; fruit or vegetable juices; bakery products, including bread, cakes, pastries, pies and cream-filled baked goods, and prepared foods, such as egg dishes, pastas and salads, including egg, tuna, chicken, potato and pasta salads. The diagnostic assays are also useful in the assessment of microbiologically pure cultures and water quality, and in environmental and pharmaceutical quality control processes.

While the primary focus of E. coli O157 detection is food products, the present invention also contemplates the use of the primers and probes in diagnostic assays for the detection of E. coli O157 contamination of other biological samples, such as patient specimens in a clinical setting, for example, faeces, blood, saliva, throat swabs, urine, mucous, and the like. The diagnostic assays are also useful in the assessment of microbiologically pure cultures, and in environmental and pharmaceutical quality control processes.

The test sample can be used in the assay either directly (i.e. as obtained from the source) or following one or more pre-treatment steps to modify the character of the sample. Thus, the test sample can be pre-treated prior to use, for example, by disrupting cells or tissue, enhancing/enriching the microbial content of the sample by culturing in a suitable medium, preparing liquids from solid materials, diluting viscous fluids, filtering liquids, distilling liquids, concentrating liquids, inactivating interfering components, adding reagents, purifying nucleic acids, and the like. In one embodiment of the present invention, the test sample is subjected to one or more steps to isolate, or partially isolate, nucleic acids therefrom. In another embodiment of the invention, the test sample is subjected to an enrichment procedure to enhance the microbial content of the sample prior to use in the assay.

As indicated above, the polynucleotide primers and probes of the invention can be used in assays to quantitate the amount of an E. coli O157 target nucleotide sequence in a test sample. Thus, the present invention provides for method to specifically amplify, detect and quantitate a target nucleotide sequence in a test sample, the methods generally comprising the steps of:
(a) forming a reaction mixture comprising a test sample, amplification reagents, at least one labelled polynucleotide probe sequence capable of specifically hybridising to a portion of an E. coli O157 target nucleotide sequence and at least one polynucleotide primer corresponding to or complementary to a portion of an E. coli O157 rfbE gene comprising said target nucleotide sequence;
(b) subjecting the mixture to amplification conditions to generate at least one copy of the target nucleotide sequence, or a nucleic acid sequence complementary to the target nucleotide sequence;
(c) hybridizing the probe to the target nucleotide sequence or the nucleic acid sequence complementary to the target sequence, so as to form a probe:target hybrid;
(d) detecting the probe:target hybrid by detecting the signal produced by the hybridized labelled probe; and
(e) analysing the amount of signal produced as an indication of the amount of target nucleotide sequence present in the test sample.

Step (e) can be conducted, for example, by comparing the amount of signal produced to a standard or utilising one of a number of statistical methods known in the art that does not require a standard.

The steps of this method may also be varied as described above for the amplification/detection method.

Various types of standards for quantitative assays are known in the art. For example, the standard can consist of a standard curve compiled by amplification and detection of known quantities of the E. coli O157 target nucleotide sequence under the assay conditions. Alternatively, relative quantitation can be performed without the need for a standard curve (see, for example, Pfaffl, M W. (2001) Nucleic Acids Research 29(9):2002-2007). In this method, a reference gene is selected against which the expression of the target gene can be compared. The reference gene is usually a gene that is expressed constitutively, for example, a house-keeping gene. An additional pair of primers and an appropriate probe are included in the reaction in order to amplify and detect a portion of the selected reference gene.

Another similar method of quantification is based on the inclusion of an internal standard in the reaction. Such internal standards generally comprise a control target nucleotide sequence and a control polynucleotide probe. The internal standard can further include an additional pair of primers that specifically amplify the control target nucleotide sequence and are unrelated to the polynucleotides of the present invention. Alternatively, the control target sequence can contain primer target sequences that allow specific binding of the assay primers but a different probe target sequence. This allows both the E. coli target sequence and the control sequence to be amplified with the same primers, but the amplicons are detected with separate probe polynucleotides. Typically, when a reference gene or an internal standard is employed, the reference/control probe incorporates a detectable label that is distinct from the label incorporated into the E.coli target sequence specific probe. The signals generated by these two labels when they bind their respective target sequences can thus be distinguished.

In the context of the present invention, a control target nucleotide sequence is a nucleic acid sequence that (i) can be amplified either by the E.coli target sequence specific primers or by control primers, (ii) specifically hybridizes to the control probe under the assay conditions and (iii) does not exhibit significant hybridization to the E.coli target sequence specific probe under the same conditions. One skilled in the art will recognise that the actual nucleic acid sequences of the control target nucleotide and the control probe are not important provided that they both meet the criteria outlined above.

The diagnostic assays can be readily adapted for high-throughput. High-throughput assays provide the advantage of processing many samples simultaneously and significantly decrease the time required to screen a large number of samples. The present invention, therefore, contemplates the use of the polynucleotides of the present invention in high-throughput screening or assays to detect and/or quantitate E. coli O157 target nucleotide sequences in a plurality of test samples.

For high-throughput assays, reaction components are usually housed in a multi-container carrier or platform, such as a multi-well microtitre plate, which allows a plurality of assays each containing a different test sample to be monitored simultaneously. Control samples can also be included in the plates to provide internal controls for each plate. Many automated systems are now available commercially for high-throughput assays, as are automation capabilities for procedures such as sample and reagent pipetting, liquid dispensing, timed incubations, formatting samples into microarrays, microplate thermocycling and microplate readings in an appropriate detector, resulting in much faster throughput times.

Kits and Packages for the Detection of E. coli O157

The present invention further provides for kits for detecting E. coli O157 in a variety of samples. In general, the kits comprise a plurality of polynucleotides capable of amplifying and/or detecting an E. coli O157 target sequence as described above. In one embodiment, the kit comprises a pair of primers and a probe capable of amplifying and detecting an E. coli O157 target sequence as described above. One of the primers and the probe may be provided in the form of a single polynucleotide, such as a Scorpion probe, as described above. The probe provided in the kit can incorporate a detectable label, such as a fluorophore or a fluorophore and a quencher, or the kit may include reagents for labelling the probe. The primers/probes can be provided in separate containers or in an array format, for example, pre-dispensed into microtitre plates.

The kits can optionally include amplification reagents, such as buffers, salts, enzymes, enzyme co-factors, nucleotides and the like. Other components, such as buffers and solutions for the enrichment, isolation and/or lysis of bacteria in a test sample, extraction of nucleic acids, purification of nucleic acids and the like may also be included in the kit. One or more of the components of the kit may be lyophilised and the kit may further comprise reagents suitable for the reconstitution of the lyophilised components. The lyophilised components may further comprise additives that facilitate their reconstitution.

The various components of the kit are provided in suitable containers. As indicated above, one or more of the containers may be a microtitre plate. Where appropriate, the kit may also optionally contain reaction vessels, mixing vessels and other components that facilitate the preparation of reagents or nucleic acids from the test sample.

The kit may additionally include one or more controls. For example, control polynucleotides (primers, probes, target sequences or a combination thereof) may be provided that allow for quality control of the amplification reaction and/or sample preparation, or that allow for the quantitation of E. coli target nucleotide sequences.

The kit can additionally contain instructions for use, which may be provided in paper form or in computer-readable form, such as a disc, CD, DVD or the like.

The present invention further contemplates that the kits described above may be provided as part of a package that includes computer software to analyse data generated from the use of the kit.

The invention will now be described with reference to specific examples. It will be understood that the following examples are intended to describe preferred embodiments of the invention and are not intended to limit the invention in any way.

### EXAMPLES

### Example 1

### Determination of Unique, Conserved DNA Regions in E. coli O157 Group

The rfbE gene coding regions from 12 different E. coli O157 isolates were sequenced and aligned using the multiple alignment program Clustal W™. The resulting alignment was used to identify short DNA regions that were conserved within the E. coli O157 group, yet which are excluded from other bacteria. FIG. 1 depicts a sample of such an alignment in which a portion of the rfbE gene of 12 different E. coli O157 isolates has been aligned. In this Figure, the E. coli O157 isolates are:
E-co-B71: E. coli serotype O157:H7 (SEQ ID NO:2)
E-co-B73: E. coli serotype O157:H7 (SEQ ID NO:3)
E-co-B74: E. coli serotype O157:H7 (SEQ ID NO:4)
E-co-B75: E. coli serotype O157:H7 (SEQ ID NO:5)
E-co-B76: E. coli serotype O157:H7 (SEQ ID NO:6)
E-co-B81: E. coli serotype O157:H7 (SEQ ID NO:7)
E-co-B83: E. coli serotype O157:H7 (SEQ ID NO:8)
E-co-B86: E. coli serotype O157:H7 (SEQ ID NO:9)
E-co-B88: E. coli serotype O157:H7 (SEQ ID NO:10)
E-co-B94: E. coli serotype O157:H7 (SEQ ID NO:11)
E-co-B96: E. coli serotype O157:H7 (SEQ ID NO:12)
E-co-B100: E. coli serotype O157:H7 (SEQ ID NO:13)

A 107 nucleotide conserved sequence was identified as described above (shown in FIG. 4B and SEQ ID NO:14). This unique and conserved element of E. coli O157 rfbE-gene sequences was used to design highly specific primers for the PCR amplification of a conserved region of the rfbE gene.

### Example 2

### Generation of PCR Primers for Amplication of the rfbE Gene Segment

Within the conserved 107 nucleotide sequence identified as described in Example 1, two regions that could serve as primer target sequences were identified. These primer target sequences were used to design a pair of primers to allow efficient PCR amplification. The primer sequences are shown below:

| | |
|---|---|
| Forward primer: | |
| 5′-AGGTGGAATGGTTGTCAC-3′ | [SEQ ID NO:16] |
| Reverse primer: | |
| 5′-AGCCTATAACGTCATGCC-3′ | [SEQ ID NO:17] |

In the alignment presented in FIG. 1, the positions of the forward and reverse primers are represented by shaded boxes. The forward primer starts at position 53 and ends at position 70 of the alignment. The reverse primer represents the reverse complement of the region starting at position 142 and ending at position 159.

### Example 3

### Generation of Molecular Beacon Probes Specific for E. coli O157

In order to design molecular beacon probes specific for E. coli O157, a region within the primer amplification region described above was identified which not only was highly conserved in all E. coli O157 isolates but was also exclusive to E. coli O157 isolates. This sequence consisted of a 28 nucleotide region that would be suitable for use as a molecular beacon target sequence. The sequence is provided below:

| | |
|---|---|
| 5′-ACCGTTGTTTACATTTTAAAGGCCAAGG-3′ | [SEQ ID NO:15] |

The complement of this sequence is also suitable for use as a molecular beacon target sequence.

A molecular beacon probe having the sequence shown below was synthesized by Integrated DNA Technologies Inc.

Molecular beacon probe #3:

[SEQ ID NO: 18]

5′-CGCACCGTTGTTTACATTTTAAAGGCCAAGGTGCG-3′

The complement of this sequence (SEQ ID NO:19, shown below) can also be used as a molecular beacon probe for the detecting E.coli O157.

[SEQ ID NO:19]

5′-CGCACCTTGGCCTTTAAAATGTAAACAACGGTGCG-3′

The starting material for the synthesis of the molecular beacons was an oligonucleotide that contains a sulfhydryl group at its 5′ end and a primary amino group at its 3′ end. DABCYL was coupled to the primary amino group utilizing an amine-reactive derivative of DABCYL. The oligonucleotides that were coupled to DABCYL were then purified. The protective trityl moiety was then removed from the 5′-sulfhydryl group and a fluorophore was introduced in its place using an iodoacetamide derivative.

An individual skilled in the art would recognize that a variety of methodologies could be used for synthesis of the preferred molecular beacon. For example, a controlled-pore glass column that introduces a DABCYL moiety at the 3′ end of an oligonucleotide has recently become available, which enables the synthesis of a molecular beacon completely on a DNA synthesizer.

Table 2 provides a general overview of the characteristics of molecular beacon probe #3. The beacon sequence shown in Table 2 indicates the stem region in lower case and the loop region in upper case. Bases marked with an * are included in both the Tm stem and Tm loop calculations given in Table 3.

TABLE 2
Description of molecular beacon probe #3.

Beacon sequence (5′ to 3′):
cgca*c*c*GTTGTTTACATTTTAAAGGCCAAg*g*tgcg
Fluorophore (5′): FAM
Quencher (3′): DABCYL

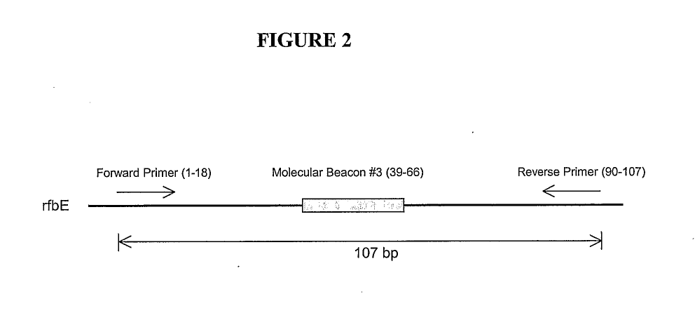

Table 3 provides an overview of the thermodynamics of the folding of molecular beacon probe #3. Calculations were made using MFOLD™ software, or the Oligo Analyzer software package available on Integrated DNA Technologies Inc. web site. FIG. 2 shows the arrangement of PCR primers and the molecular beacon probe in the rfbE consensus sequence. Numbers in parentheses indicate the positions of the first and last nucleotides of each feature on the PCR product generated with the forward and reverse primers.

TABLE 3
Thermodynamics of molecular beacon probe #3.

| | | |
|---|---|---|
| Tm loop (thermodynamics algorithm) | 59.1° | C. |
| Tm stem (mFOLD calculation) | 64.2° | C. |
| ΔG₃₇ (mFOLD calculation) | −4.1 | kCal/mol |
| ΔH (mFOLD calculation) | −50.4 | kCal/mol |

Two other molecular beacons suitable for the detection of E. coli O157 were also prepared as described above. The sequences are shown below (nucleotides in lower case represent the nucleotides that make up the stem of the beacon):

Molecular beacon probe #1:

[SEQ ID NO:22]

5′-cgcgcCCGTTGTTTACATTTTAAAGGCCgcgcg-3′
Molecular beacon probe #2:

[SEQ ID NO:25]

5′-cgagcgCCGTTGTTTACATTTTAAAGGCCcgctcg-3′

The complement of these sequences (SEQ ID NOs:24 and 26, respectively, see below) can also be used as molecular beacon probes for the detection of E. coli O157.

[SEQ ID NO:24]

5′-cgcgcGGCCTTTAAAATGTAAACAACGGgcgcg-3′

[SEQ ID NO:26]

5′-cgagcgGGCCTTTAAAATGTAAACAACGGcgctcg-3′

### Example 4

### Isolation of DNA from Samples

The following protocol can be utilized in order to isolate DNA sequences from samples.

Materials required for DNA extraction:
Tungsten carbide beads: Qiagen
Reagent DX: Qiagen
DNeasy Plant Mini Kit: Qiagen
Tissue Disruption equipment: Mixer Mill™ 300 (Qiagen)

Methodology:
1) Add to a 2 ml screw top tube: 1 tungsten carbide bead and 0.1 g glass beads 212 to 300 μm in width+sample to be analysed+500 μL of AP1 buffer+1 μL of Reagent DX+1 μL of RNase A (100 mg/mL). Extraction control was performed without adding sample to be analysed.
2) heat in Dry-Bath at 80° C. for 10 min.
3) mix in a Mixer Mill 300 (MM300) at frequency of 30 Hz [1/s], 2 min.
4) rotate tubes and let stand for 5 min at room temperature.
5) mix in a Mixer Mill 300, frequency 30 Hz, 1 min.
6) place tubes in boiling water for 5 min.
7) centrifuge with a quick spin.
8) add 150 μL of AP2 buffer.
9) mix at frequency of 30 Hz for 30 sec. Rotate tubes and repeat.
10) centrifuge at 13,000 rpm for 1 min.
11) transfer supernatant in to a 2 mL screw top tube containing 800 μL of AP3/E buffer.
12) mix by inverting, centrifuge with a quick spin.
13) add 700 μL of mixture. From step 13 to a DNeasy binding column and centrifuge at 800 rpm for 1 minute. Discard eluted buffer. Repeat process with leftover mixture from step 13.
14) add 500 μL of wash buffer (AW buffer) to binding columns and centrifuge for 1 minute at 800 rpm. Discard eluted buffer.
15) add 500 μL of wash buffer (AW buffer) to binding columns and centrifuge for 1 minute at 800 rpm. Discard eluted buffer.
16) centrifuge column again at 8000 rpm for 1 min.
17) place column in a sterile 2 mL tube and add 100 μL of AE elution buffer preheated at 80° C.
18) incubate for 1 min. Centrifuge at max speed for 2 min. Elute twice with 100 μL.
19) keep elution for PCR amplification.

Time of manipulation: 3 hours. Proceed to prepare PCR reaction for real-time detection.

### Example 5

### Amplification of a Target Sequence and Hybridization of Molecular Beacon Probe #3 in Real Time

PCR amplification was undertaken using the conditions described in Tables 4 and 5 below. The intensity of fluorescence emitted by the fluorophore component of the molecular beacon was detected at the annealing stage of each amplification cycle. In Table 4, note that the PCR buffer contains 2.25 mM magnesium chloride (final concentration). Inclusion of additional magnesium chloride brings the final concentration to 4 mM in the reaction mixture.

TABLE 4
PCR mix used for validation

| | Final concentration in | |
|---|---|---|
| Reagent | reconstituted reaction | |
| Qiagen PCR buffer, 10X | 1.5X | |
| Forward primer, 25 μM | 0.5 | μM |
| Reverse primer, 25 μM | 0.5 | μM |
| dNTPs, 10 mM | 0.2 | mM |
| MgCl₂, 25 mM | 1.75 | mM |
| Molecular beacon #3, 10 μM | 0.3 | μM |

| | |
|---|---|
| HotStarTaq, 5 U/μL | 1 U/25 μL reaction |

Table 5 presents an overview of the cycles used for each step of the PCR amplification.

TABLE 5
PCR program used throughout diagnostic test validation.

| Step | Temperature | Duration | | Repeats |
|---|---|---|---|---|
| Initial polymerase activation | 95° C. | 15 | min | 1 |
| Denaturation | 94° C. | 15 | sec | 40 |
| Annealing | 55° C. | 15 | sec | |
| Elongation | 72° C. | 15 | sec | |

Fluorescence was detected in real-time using a fluorescence monitoring real-time PCR instrument, for example, a BioRad iCycler iQ™ or MJ Research Opticon™. Other instruments with similar fluorescent reading abilities can also be used.

### Example 6

### Quantification of Target Sequence in a Sample

In order to quantify the amount of target sequence in a sample, DNA was isolated and amplified as described in the preceding Examples (4 and 5). DNA was quantified using a standard curve constructed from serial dilutions of a target DNA solution of known concentration.

### Example 7

### Positive Validation for the Specificity of Molecular Beacon Probe #3 for Detection of E. coli O157

The effectiveness of molecular beacon probe #3 for detecting E. coli O157 isolates was demonstrated as described generally below.

Genomic DNA from the species and strains presented in Table 6 below was isolated and amplified as described in Example 5. Results are presented in Table 6 and indicate that molecular beacon probe #3 was capable of detecting all E. coli O157 isolates tested. In Table 6, figures in parentheses indicate the number of strains of each O157 serotype that were tested (if more than one). All strains gave a positive signal.

TABLE 6
Positive validation of molecular beacon probe #3
and forward and reverse primers.

| | | | |
|---|---|---|---|
| Escherichia coli | Escherichia coli | Escherichia | Escherichia coli |
| O157:H19 | O157:H7 (51) | coli | O157:HNM (2) |
| | | O157:H43 (3) | |
| Escherichia coli | Escherichia coli | | |
| O157 | O157:NM (12) | | |

### Example 8

### Negative Validation of the Primers and Molecular Beacon Probe #3

In order to test the ability of molecular beacon probe #3 to preferentially detect only E. coli O157, 241 bacterial strains from groups other than E. coli O157 were tested, including 203 non-O157 E. coli strains, as generally described below.

Samples of genomic DNA from the bacteria presented in Table 7 below were isolated and amplified using conditions and parameters as described in Example 5. No hybridization of this molecular beacon was observed.

In Table 7, the figures in parentheses indicate the number of strains of each species that were tested (if more than one). None of the tested strains provided a positive result.

The results presented in Table 7 indicate that the amplification primers and the molecular beacon are highly specific for E. coli O157.

TABLE 7
Negative Validation of the Primers and Molecular Beacon probe #3

| | | | |
|---|---|---|---|
| Acinetobacter | E. coli | E. coli | Neisseria lactamica |
| calcoaceticus (2) | O111:HN | O7:H21 (2) | |
| Acinetobacter iwoffi | E. coli | E. coli | Neisseria meningitidis |
| | O111:HNM | O7:NM (5) | (2) |
| Acinetobacter junii | E. coli | E. coli | Neisseria sica |
| | O111:NM | O71:H12 | |
| Aeromonas | E. coli | E. coli | Nocardia asteroides |
| hydrophila (2) | O111A:HNM | O75:H5 | |
| Aeromonas | E. coli | E. coli | Pediococcus acidilactici |
| salmonicida (2) | O112:H18 | O75:NM | (2) |
| Alcaligenes faecalis | E. coli | E. coli | Pediococcus |
| | O112:H8 | O77:NM | pentosaceus |
| Bacillus | E. coli | E. coli | Proteus mirabilis (2) |
| amyloliquefaciens | O112a, 112c:K66(b11): | O78:H11 | |
| (2) | NM | | |
| Bacillus cereus (2) | E. coli | E. coli | Proteus penneri (2) |
| | O112AC:NM | O78:NM (3) | |
| Bacillus circulans | E. coli | E. coli | Proteus vulgaris (2) |
| (2) | O113:H21(2) | O79:H25 | |
| Bacillus coagulans | E. coli | E. coli | Pseudomonas |
| (2) | O114:H32 | O79:H43 | aeruginosa (2) |
| Bacillus firmus | E. coli | E. coli | Pseudomonas |
| | O117:H4 | O79:NM | alcaligenes |
| Bacillus lentus | E. coli | E. coli | Pseudomonas |
| | O119:H18 | O8:H9 | mendocina |
| Bacillus | E. coli | E. coli O80:H26 | Pseudomonas |
| licheniformis (2) | O119:K69(b14) | | pseudoalcaligenes |
| Bacillus megaterium | E. coli | E. coli | Pseudomonas putida (2) |
| (2) | O12:NM | O85:HN | |
| Bacillus myoides | E. coli | E. coli | Pseudomonas stutzeri |
| | O121:HN | O86:H43 | |
| Bacillus pumilus (2) | E. coli | E. coli | Salmonella bongori |
| | O124:H25 | O86:NM (3) | |
| Bacillus sphaericus | E. coli | E. coli | Salmonella choleraesuis |
| | O125ab:H19 | O88:NM | (2) |
| Bacillus | E. coli | E. coli | Salmonella choleraesuis |
| stearothermophilus | O126:H2 | O89:HN | subsp. Arizonae (2) |
| Bacillus subtilis (2) | E. coli | E. coli | Salmonella enterica |
| | O127:K63(b8) | O9:H12 | subsp. indica |
| Bacillus | E. coli | E. coli | Salmonella enterica |
| thuringiensis (2) | O127:NM | OM:H18 | subsp. enterica serovar |
| | | | Dublin (2) |
| Bacteroides fragilis | E. coli | E. coli | Salmonella enterica |
| | O128:H2 (2) | OM:HN | subsp. enterica serovar |
| | | | Infantis (2) |
| Bifidobacterium | E. coli | E. coli | Salmonella enterica |
| adolescentis | O128:H21 (3) | ON:H10 (2) | subsp. enterica serovar |
| | | | Montevideo (2) |
| Bifidobacterium | E. coli | E. coli | Salmonella enterica |
| animalis | O128:H47 | ON:H26 | subsp. enterica serovar |
| | | | Newport (2) |
| Bifidobacterium | E. coli | E. coli | Salmonella enterica |
| bifidum | O128:H7 (4) | ON:H32 | subsp. enterica serovar |
| | | | Saintpaul (2) |
| Bifidobacterium | E. coli | E. coli | Salmonella enterica |
| longum | O128:HNM | ON:HM (2) | subsp. enterica serovar |
| | | | Senftenberg |
| Bifidobacterium | E. coli | E. coli | Salmonella enterica |
| pseudolongum | O128a:H2 (2) | ON:HN (8) | subsp. enterica serovar |
| | | | Stanley |
| Bifidobacterium spp. | E. coli | E. coli | Salmonella enterica |
| (2) | O128a:H21 | ON:NM (6) | subsp. enterica serovar |
| | | | Thompson (2) |
| Bifidobacterium suis | E. coli | E. coli | Salmonella enterica |
| | O128AB:H2 | Serotype B | subsp. enterica serovar |
| | | | Typhisuis (2) |
| Bifidobacterium | E. coli | Edwardsiella | Salmonella enterica, |
| thermophilus | O128AB:H8 | tarda | subsp. diarizonae |
| Bordetella | E. coli | Enterobacter | Salmonella enterica, |
| bronchiseptica | O128AC:NM | aerogenes (2) | subsp. enterica serovar |
| | | | Agona |
| Bordetella pertussis | E. coli | Enterobacter | Salmonella enterica, |
| | O13:NM | amnigenus | subsp. enterica serovar |
| | | | Brandenburg |
| Borrelia burgdorferi | E. coli | Enterobacter | Salmonella enterica, |
| | O136:NM | cloacae (2) | subsp. enterica serovar |
| | | | Heidelberg (2) |
| Branhamella | E. coli | Enterobacter | Salmonella enterica, |
| catarrhalis | O14:NM | intermedius (2) | subsp. houtenae |
| Brevibacillus | E. coli | Enterobacter | Salmonella enteritidis |
| laterosporus | O144:H8 | taylorae | (2) |
| Campylobacter coli | E. coli | Enterococcus | Salmonella paratyphi (4) |
| | O15:NM (2) | faecalis (2) | |
| Campylobacter | E. coli | Enterococcus | Salmonella typhi (2) |
| jejuni (2) | O150:H21 | faecium | |
| Campylobacter lari | E. coli | Enterococcus | Salmonella typhimurium |
| (2) | O18:H14 (2) | hirae (2) | (2) |
| Campylobacter | E. coli | Erwinia | Serratia liquefaciens (2) |
| rectus | O18AC:NM | herbicola | |
| Cellilomonea spp. | E. coli | Escherichia | Serratia marcescens (2) |
| | O2:HN | fergusonii | |
| Chromobacterium | E. coli | Escherichia | Serratia odorifera |
| violaceum | O2:NM (3) | hermanii (3) | |
| Chryseobacterium | E. coli | Escherichia | Shigella boydii |
| spp. | O23:H15 | vulneris (3) | |
| Chryseomonas | E. coli | Haemophilus | Shigella dysenteriae (2) |
| luteola | O24:NM (2) | equigenitalis | |
| Citrobacter | E. coli | Haemophilus | Shigella flexneri (2) |
| amalonaticus (2) | O25:H1 (2) | influenzae (2) | |
| Citrobacter diversus | E. coli | Haemophilus | Shigella sonnei (2) |
| | O25:H2 | paragallinarum | |
| Citrobacter freundii | E. coli | Hafnia alvei (2) | Staphylococcus aureus |
| (2) | O25:HN | | (2) |
| Citrobacter koseri | E. coli | Helicobacter | Staphylococcus |
| (2) | O25:NM | pylori | chromogenes |
| Citrobacter | E. coli | Klebsiella | Staphylococcus |
| werkmanii | O26:H11 (8) | ornithinolytica | epidermidis (2) |
| Clostridium | E. coli | Klebsiella | Staphylococcus |
| botulinum (2) | O26:NM | oxytoca (2) | intermedius |
| Clostridium | E. coli | Klebsiella | Staphylococcus lentis |
| butyricum | O28:NM | planticola (2) | |
| Clostridium difficile | E. coli | Klebsiella | Staphylococcus |
| | O3:H44 | pneumoniae (2) | ludgdunensis |
| Clostridium | E. coli | Klebsiella | Staphylococcus |
| perfringens (2) | O3:K2a, bb(1):H2 | terrigena | schieiferi |
| Clostridium | E. coli | Kocuria kristinae | Staphylococcus xylosus |
| sporogenes | O36:H9 | | |
| Clostridium tetani | E. coli | Kurthia zopfii (2) | Stenotrophomonas |
| | O4:H40 (2) | | maltophilia |
| Clostridium | E. coli | Lactobacillus | Streptococcus |
| tyrobutyricum | O4:H43 | acidophilus | agalactiae (2) |
| Corynebacterium | E. coli | Lactobacillus | Streptococcus bovis |
| xerosis | O4:H5 | casei (2) | |
| E. blattae (3) | E. coli | Lactobacillus | Streptococcus |
| | O4:HN (2) | delbreuckii (2) | pneumoniae (2) |
| E. coli (9) | E. coli | Lactobacillus | Streptococcus pyogenes |
| | O40:H(NT) | helveticus | (2) |
| E. coli | E. coli | Lactobacillus | Streptococcus suis |
| O1:H6 | O44:H18 | pentosus | |
| E. coli | E. coli | Lactobacillus | Streptococcus |
| O1:H7 | O44:H23 | plantarum (2) | thermophilus |
| E. coli | E. coli | Lactobacillus | Vibrio alginolyticus |
| O1:NM (3) | O45:H2 | rhamnosus (2) | |
| E. coli | E. coli | Lactococcus | Vibrio cholerae (2) |
| O10:K5(1):H4 | O5:H4 | lactis (2) | |
| E. coli | E. coli | Lactococcus | Vibrio eltor |
| O10:NM | O5:HN | raffinolactis | |
| E. coli | E. coli | Legionella | Vibrio fluvialis |
| O102:H40 | O5:NM | pneumophila (2) | |
| E. coli | E. coli | Listeria grayi | Vibrio hollisae |
| O104:H21 | O50:H4 | | |
| E. coli | E. coli | Listeria innocua | Vibrio vulnificus |
| O104:NM (2) | O55:H5 | (2) | |
| E. coli | E. coli | Listeria ivanovii | Xanthomonas |
| O106:NM | O55:H6 (9) | (2) | campestris |
| E. coli | E. coli | Listeria | Yersinia enterocolitica |
| O111:H11 | O55:H7 (10) | monocytogenes | (2) |
| | | (2) | |
| E. coli | E. coli | Listeria seeligeri | Yersinia frederiksenii |
| O111:H12 (3) | O55:NM (2) | | |
| E. coli | E. coli | Listeria | Yersinia kritensenii |
| O111:H2 (3) | O6:H1 (2) | welshimeri (2) | |
| E. coli | E. coli | Micrococcus | |
| O111:H21 (6) | O6:H10 (2) | luteus (2) | |
| E. coli | E. coli | Mycobacterium | |
| O111:H8 (2) | O6:H49 | smegmatis | |
| E. coli | E. coli | Neisseria | |
| O111:HM | O62:H32 | gonorrhoeae | |

### Example 9

### Validation of Molecular Beacon Probes #1 and #2

Specificity studies were performed using molecular beacon probes #1 and #2 (SEQ ID NOs:22 and 25, respectively) using similar target and non target layouts as described above (Examples 7 and 8), with the exception that fewer bacterial strains were tested (26 E. coli O157 strains, 115 non-E. coli O157 strains and 251 non-E. coli strains). The results showed that molecular beacons #1 and #2 were also 100% specific to E. coli O157.

### Example 10

### Sensitivity of Molecular Beacon Probes #1, #2 and #3

The three molecular beacon probes (#1, #2 and #3) were tested with a gradient of E. coli DNA that had been extracted using the Qiagen extraction kit. All three beacons were able to detect a 10⁻⁴ DNA dilution (initial concentration unknown). Parameters were as follows:
Molecular beacon #1: Cₜ 36 (for 10⁻⁴ dilution), highest RFU 400.
Molecular beacon #2: Cₜ 36.25 (for 10⁻⁴ dilution), highest RFU 300.
Molecular beacon #3: Cₜ 34.8 (for 10⁻⁴ dilution), highest RFU 500.

The sensitivity of the three molecular beacons was also tested using DNA extracted from ground pork (25 g ) spiked with 2 CFU of E coli O157 per gram after 19 hours of enrichment. Parameters were as follows:
Molecular beacon #1: 10⁻³ dilution detected at Cₜ 36.73 using Ct calculator; highest RFU ˜500.
Molecular beacon #2: 10⁻³ dilution detected at Cₜ 37.45 using Ct calculator; highest RFU ˜400.
Molecular beacon #3: 10⁻⁴ dilution detected at Cₜ 36.70 using Ct calculator; highest RFU ˜1000.

From experiments using pure cultures of E. coli, it has been determined that an observed Ct of 36-39 corresponds to detection of one copy of the genome.

### Example 11

### Performance of Molecular Beacon Probes #1, #2 and #3

The performance of molecular beacons #1, #2 and #3 with the forward and reverse primers described in Example 2[SEQ ID NOs:16 and 17] was assessed relative to a beacon sequence (O157rfbE) and a pair of primers O157BF and O157BR) described previously (Fortin et al., (2001) Analytical Biochem. 289:281-288).

Probe O157rfbE:

[SEQ ID NO:27]

5′-CGCTATGGTGAAGGTGGAATGGTTGTCACGAATAGCG-3′
Primer O157BF:

[SEQ ID NO:28]

5′-AAATATAAAGGTAAATATGTGGGAACATTTGG-3′
Primer O157BR:

[SEQ ID NO:29]

5′-TGGCCTTTAAAATGTAAACAACGGTCAT-3′

The efficiency of the primers and probes was assessed against two strains of E. coli O157 (B74 and B76) using two sets of PCR conditions, as described below. The specificity of the primers and probes was also tested using over 200 non-O157 E. coli strains and over 200 other non-E. coli bacteria.

### PCR Conditions A:

For each PCR, 2 μl of template DNA was added to 23 μl of PCR master mix (1.75 mM MgCl₂, 0.5 μM of each primers, 0.2 mM dNTPs mix, 0.3 μM Beacon, 0.04 U/μl HotStarTaq, 1.5× PCR buffer (containing 15 mM MgCl₂), and water). The final reaction contains 4 mM MgCl₂. All reactions were performed in 200 μl 96 well plates (BioRad) sealed with the Optical Tape (BioRad). The iCycler BioRad system was used for real-time analyses.

For the PCR reactions, samples were heated at 95° C. for 13:30 min, followed by 40 cycles of melting at 94° C. for 15 s, fluorescent measurement at 55° C. for 15 s (annealing), and extension at 72° C. for 15 s. At the end of each PCR run, data were automatically analyzed, amplification plots were obtained and the threshold cycle (Cₜ) calculated.

### PCR Conditions B:

For each PCR, 2.5 μl of template DNA was added to 27.5 μl of PCR master mix (3.5 mM MgCl₂, 0.5 μM of each primers, 0.2 μM dNTPs mix, 1 μM MB, 5 U AmpliTaq Gold DNA polymerase, 2.5 μl TaqMan buffer A, and the remainder water). Both the polymerase and the amplifying buffer A were purchased as part of the TaqMan PCR core reagent kit (PE Biosystems, Foster City, Calif.). All reactions were performed in the 200 μl MicroAmp optical tubes sealed with the MicroAmp optical caps (PE Biosystems). The Pelkin-Elmer ABI Prism 7700 sequence detection system was used for real-time analyses.

For the PCR reactions, samples were heated at 95° C. for 10 min, followed by 40 cycles of melting at 94° C. for 45 s, fluorescent measurement at 41° C. for 30 s, annealing at 52° C. for 45 s, and extension at 72° C. for 45 s. At the end of each PCR run, data were automatically analyzed, amplification plots were obtained and the threshold cycle (Cₜ) of each amplification reaction was calculated based on the first PCR cycle at which the fluorescence was 10-fold higher than the standard deviation of the mean baseline emission.

Table 8 presents the results of comparisons of the primer pairs using PCR conditions A described above, in the absence of a molecular beacon probe, and two different E. coli O157 isolates. SYBR Green was used to detect the product of the amplification reaction.

TABLE 8
Comparison of Primer Pairs

| | Primer pair | |
|---|---|---|
| Test | SEQ ID NOs: 28 & 29 | SEQ ID NOs: 16 & 17 |
| Primer | 6 dilutions² detected for both | 6 dilutions² detected for both |
| efficiency¹ | strains; efficiencies were 96.3% | strains; efficiencies were 99.1% |
| | (B74) and 101.9% (B76). | (B74) and 106.8% (B76). |
| Temperature | For both strains, primers were | For both strains, primers were |
| gradient | most stable at 62.1° C. | most stable at 59.3° C. |
| Specificity: | All E. coli O157 amplified. RFU | All E. coli O157 amplified. RFU |
| E. coli O157 | between 200-350. | between 550-850. |
| vs. E. coli | | |
| Specificity: | No cross-amplification. | One Yersinia strain amplified |
| other bacteria | | with a Cₜ of 30.5³. |

¹90% efficiency or greater is optimal to prevent the PCR reaction being limited by any single reaction component.
²This represents a limit of detection of 2 genomes. The starting concentration was 0.5 ng/μl and 2 μl are added to each PCR reaction for a total of 1 ng, which corresponds to 200,000 genomes.
³The melting point indicated that a different product had been amplified (Tm = 83 instead of 80).

The RFU readings for the two sets of primers indicate that, under the conditions used, amplification of E. coli O157 nucleic acids with primer pair SEQ ID NOs:16 & 17 gave higher fluorescence readings than amplification with primer pair SEQ ID NOs:28 & 29.

Table 9 presents the results of a comparison of the molecular beacons using PCR conditions A as described above and two different E. coli O157 isolates. For this test, the molecular beacon O157 rfbE [SEQ ID NO:27] was tested with primer pair SEQ ID NOs:28 & 29 and molecular beacons #1[SEQ ID NO:22], #2[SEQ ID NO:25] and #3[SEQ ID NO:18] were tested with primer pair SEQ ID NOs:16 & 17.

TABLE 9
Comparison of Beacon Probes under PCR conditions A

| | Probe | | | |
|---|---|---|---|---|
| Test | O157rfbE | Beacon #1 | Beacon #2 | Beacon #3 |
| Signal/Noise¹ | 6.31 | 11.06 | 27 | 28 |
| Molecular | 5 dilutions² | 6 dilutions³ | 6 dilutions³ | 5 dilutions² |
| beacon | detected for | detected for | detected for | detected for |
| efficiency | both strains; | both strains; | both strains; | both strains; |
| | efficiencies of | efficiencies of | efficiencies of | efficiencies of |
| | 111.7% (B74) | 93.3% (B74) | 104.3% (B74) | 105% (B74) |
| | and 109.6% | and 100.74% | and 97.4% | and 101.7% |
| | (B76). | (B76). | (B76). | (B76). |
| Specificity: | All E. coli | All E. coli | All E. coli | All E. coli |
| E. coli O157 | O157 | O157 | O157 | O157 |
| vs. E. coli | amplified and | amplified and | amplified and | amplified and |
| | detected | detected | detected | detected |
| Specificity: | No cross- | Not | Not | No cross- |
| other bacteria | amplification | determined⁴ | determined⁴ | amplification |
| | or detection | | | or detection |

¹measures molecular beacon design efficiency.
²This represents a limit of detection of 20 genomes. The starting concentration was 0.5 ng/μl and 2 μl were added to each PCR reaction for a total of 1 ng, which corresponds to 200,000 genomes.
³This represents a limit of detection of 2 genomes with a starting DNA amount of 1 ng (corresponding to 200,000 genomes).
⁴As the loop sequences of Beacons #1 and #2 are the very similar to that of Beacon #3, it is assumed that these Beacons also will not cross-amplify or detect nucleic acids from other bacteria.

For the specificity tests in which the specificity of the beacons against E. coli O157 was compared to the specificity against non-O157 E. coli strains, the amplification curves for the O157rfbE beacon were not as smooth as those for beacons #1, #2 or #3. Amplification reactions that included beacon #3 typically reached the Cₜ one or two cycles prior to those that included beacon O157rfbE (e.g. 23.7 vs. 25), i.e. molecular beacon #3 demonstrated a higher sensitivity than beacon O157rfbE. In addition, the RFU for beacon O157rfbE were about 200 whereas for beacon #3 the RFU were about 300.

The beacons and primers were also assessed using the PCR conditions B outlined above. Under these conditions, the beacon O157rfbE gave a RFU approximately 3 times greater than the RFU for beacon #3.

The above results indicate that the molecular beacon probes #1, #2 and #3 perform with greatest efficiency under PCR conditions A, whereas beacon O157rfbE performs with greatest efficiency under PCR conditions B.

PCR conditions A provide for a PCR assay that can be completed in a time of between 1.5 hours and 1.75 hours (as compared to approximately 3.25 hours for assays utilising PCR conditions B), therefore, the ability of molecular beacon probes #1, #2 and #3 to perform more efficiently under PCR conditions A represents a significant advantage over beacon O157rfbE in terms of rapid real-time detection of E. coli O157.

### Example 12

### Enrichment Procedure for Test Samples

Samples to be tested can be enriched prior to use in the assay using standard enrichment procedures. The following is representative protocol for food samples.
1) Place 25 g or 25 ml of the sample in a stomacher filter bag with 225 mL of Tryptic Soy Broth (TSB) to make a 1:10 dilution.
2) Homogenize the contents of the bag for 10 sec, or until homogeneous, using a Stomacher instrument (BagMixer).
3) Incubate the stomacher bag at 35° C. for 18-24 hours in a storage rack with a closure clip attached to bag.
4) After incubation, shake to stomacher bag to homogenise the content.
5) Transfer 1 mL of the cell suspension in the bag (taking care not to take samples from the side of the stomacher bag that contains food particles) to a 2 mL sterile tube and proceed with DNA extraction (for example, following the protocol in Example 4).

Although the invention has been described with reference to certain specific embodiments, various modifications thereof will be apparent to those skilled in the art without departing from the spirit and scope of the invention as outlined in the claims appended hereto.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

1. A combination of polynucleotides for the amplification and detection of a portion of an E. coli O157 rfbE gene, said portion being less than about 475 nucleotides in length and comprising at least 65 consecutive nucleotides of the sequence set forth in SEQ ID NO:14, said combination of polynucleotides comprising:
(a) a first polynucleotide primer comprising at least 7 consecutive nucleotides of the sequence as set forth in SEQ ID NO:1;
(b) a second polynucleotide primer comprising at least 7 consecutive nucleotides of a sequence complementary to SEQ ID NO:1; and
(c) a polynucleotide probe comprising at least 7 consecutive nucleotides of the sequence as set forth in SEQ ID NO:14, or the complement thereof.

2. The combination of polynucleotides according to claim 1, wherein said portion of the E. coli O157 rfbE gene comprises the sequence set forth in SEQ ID NO:15.

3. The combination of polynucleotides according to claim 1, wherein said portion of the E. coli O157 rfbE gene comprises the sequence set forth in SEQ ID NO:14.

4. The combination of polynucleotides according to claim 1, wherein said first polynucleotide primer comprises at least 7 consecutive nucleotides of the sequence as set forth in any one of SEQ ID NOs:2-13 and said second polynucleotide primer comprises at least 7 consecutive nucleotides of a sequence complementary to any one of SEQ ID NOs:2-13.

5. The combination of polynucleotides according to claim 1, wherein said first polynucleotide primer comprises at least 7 consecutive nucleotides of the sequence as set forth in SEQ ID NO:14 and said second polynucleotide primer comprises at least 7 consecutive nucleotides of a sequence complementary to SEQ ID NO:14.

6. The combination of polynucleotides according to claims 1, wherein said polynucleotide probe comprises at least 7 consecutive nucleotides of the sequence as set forth in SEQ ID NO:15.

7. The combination of polynucleotides according to claim 1, wherein said polynucleotide probe is a molecular beacon probe.

8. The combination of polynucleotides according to claim 1, wherein said polynucleotide probe further comprises a fluorophore, a quencher, or a combination thereof.

9. A pair of polynucleotide primers for amplification of a portion of an E. coli O157 rfbE gene, said portion being less than about 475 nucleotides in length and comprising at least 65 consecutive nucleotides of the sequence set forth in SEQ ID NO:14, said pair of polynucleotide primers comprising:
(a) a first polynucleotide primer comprising at least 7 consecutive nucleotides of the sequence as set forth in SEQ ID NO:1; and
(b) a second polynucleotide primer comprising at least 7 consecutive nucleotides of a sequence complementary to SEQ ID NO:1.

10. The pair of polynucleotide primers according to claim 9, wherein said portion of the E. coli O157 rfbE gene comprises the sequence set forth in SEQ ID NO:15.

11. The pair of polynucleotide primers according to claim 9, wherein said portion of the E. coli O157 rfbE gene comprises the sequence set forth in SEQ ID NO:14.

12. The pair of polynucleotide primers according to claim 9, wherein said first polynucleotide primer comprises at least 7 consecutive nucleotides of the sequence as set forth in any one of SEQ ID NOs:2-13 and said second polynucleotide primer comprises at least 7 consecutive nucleotides of a sequence complementary to any one of SEQ ID NOs:2-13.

13. The pair of polynucleotide primers according to claim 9, wherein said first polynucleotide primer comprises at least 7 consecutive nucleotides of the sequence as set forth in SEQ ID NO:14 and said second polynucleotide primer comprises at least 7 consecutive nucleotides of a sequence complementary to SEQ ID NO:14.

14. The pair of polynucleotide primers according to claim 9, wherein said first polynucleotide primer comprises at least 7 consecutive nucleotides of the sequence as set forth in SEQ ID NO:16 and said second polynucleotide primer comprises at least 7 consecutive of the sequence as set forth in SEQ ID NO:17.

15. A method of detecting E. coli O157 in a sample, said method comprising:
(a) providing a test sample suspected of containing, or known to contain, E. coli O157 nucleic acids; and
(b) contacting said test sample with the combination of polynucleotides according to claim 1 under conditions that permit amplification and detection of a portion of an E. coli O157 rfbE gene,
wherein detection of said a portion of the E. coli O157 rfbE gene indicates the presence E. coli O157 in the sample.

16. The method according to claim 15, further comprising a step to enrich the microbial content of the test sample prior to step (a).

17. A kit for the detection of an E. coli O157 rfbE target sequence, said target sequence being less than about 475 nucleotides in length and comprising at least 65 consecutive nucleotides of the sequence set forth in SEQ ID NO:14, said kit comprising:
(a) a first polynucleotide primer comprising at least 7 consecutive nucleotides of the sequence as set forth in SEQ ID NO:1;
(b) a second polynucleotide primer comprising at least 7 consecutive nucleotides of a sequence complementary to SEQ ID NO:1; and
(c) a polynucleotide probe comprising at least 7 consecutive nucleotides of the sequence as set forth in SEQ ID NO:14, or the complement thereof.

18. The kit according to claim 17, wherein said target sequence comprises the sequence set forth in SEQ ID NO:15.

19. The kit according to claim 17, wherein said target sequence comprises the sequence set forth in SEQ ID NO:14.

20. The kit according to claim 17, wherein said first polynucleotide primer comprises at least 7 consecutive nucleotides of the sequence as set forth in any one of SEQ ID NOs:2-13 and said second polynucleotide primer comprises at least 7 consecutive nucleotides of a sequence complementary to any one of SEQ ID NOs:2-13.

21. The kit according to claim 17, wherein said first polynucleotide primer comprises at least 7 consecutive nucleotides of the sequence as set forth in SEQ ID NO:14 and said second polynucleotide primer comprises at least 7 consecutive nucleotides of a sequence complementary to SEQ ID NO:14.

22. The kit according to claim 17, wherein said polynucleotide probe comprises at least 7 consecutive nucleotides of the sequence as set forth in SEQ ID NO:15.

23. The kit according to claim 17, wherein said probe is a molecular beacon probe.

24. The kit according to claim 17, wherein said probe further comprises a fluorophore, a quencher, or a combination thereof.

25. An isolated E. coli O157 specific polynucleotide having the sequence as set forth in SEQ ID NO:14, or the complement thereof.

26. A polynucleotide primer of between 7 and 100 nucleotides in length for the amplification of a portion of an E. coli O157 rfbE gene, said polynucleotide primer comprising at least 7 consecutive nucleotides of the sequence as set forth in SEQ ID NO:14, or the complement thereof, with the proviso that the primer is other than SEQ ID NO:29.

27. The polynucleotide primer according to claim 26, wherein said polynucleotide primer comprises at least 7 consecutive nucleotides of the sequence as set forth in any one of: SEQ ID NOs:15, 16, 17, 20, 21 or 23.

28. The polynucleotide primer according to claim 26, wherein said polynucleotide primer comprises at least 7 consecutive nucleotides of the sequence as set forth in any one of: SEQ ID NOs:16 or 17.

29. The polynucleotide primer according to claim 28, wherein said polynucleotide primer comprises the sequence as set forth in SEQ ID NO:16 or 17.

30. A polynucleotide probe of between 7 and 100 nucleotides in length for detection of E. coli O157 nucleic acids, said polynucleotide probe comprising at least 7 consecutive nucleotides of the sequence as set forth in SEQ ID NO:14, or the complement thereof, with the proviso that the probe is other than SEQ ID NO:27.

31. The polynucleotide probe according to claim 30, wherein said probe comprises at least 7 consecutive nucleotides of the sequence as set forth in SEQ ID NO:15, or the complement thereof.

32. The polynucleotide probe according to claim 30, wherein said polynucleotide comprises the sequence as set forth in any one of SEQ ID NOs:15, 20, 21 or 23.

33. The polynucleotide probe according to claim 30, wherein said probe is a molecular beacon probe.

34. The polynucleotide probe according to claim 33, wherein said molecular beacon probe comprises a sequence as set forth in any one of SEQ ID NOs:18, 19, 22, 24, 25 or 26.

35. The polynucleotide probe according to claim 30, wherein said probe further comprises a fluorophore, a quencher, or a combination thereof.

36. A method of detecting E. coli O157 nucleic acids in a sample, said method comprising:
(a) contacting a test sample suspected of containing, or known to contain, E. coli O157 nucleic acids with the polynucleotide probe according to claim 30 under conditions that permit hybridisation of said probe to said E. coli O157 nucleic acids to form a probe:target hybrid, and
(b) detecting any probe:target hybrid,
wherein detection of said probe:target hybrid is indicative of the presence of said E. coli O157 nucleic acids in said sample.

37. A method of amplifying an E. coli O157 target nucleic acid sequence, said method comprising:
(a) forming a reaction mixture comprising a test sample suspected of containing, or known to contain, an E. coli O157 target nucleic acid sequence, amplification reagents, and the pair of polynucleotide primers according to claim 9; and
(b) subjecting the mixture to amplification conditions to generate at least one copy of said target nucleic acid sequence.

38. The combination of polynucleotides according to claim 4 wherein said polynucleotide probe comprises at least 7 consecutive nucleotides of the sequence as set forth in SEQ ID NO:15.

39. The combination of polynucleotides according to claim 5, wherein said polynucleotide probe comprises at least 7 consecutive nucleotides of the sequence as set forth in SEQ ID NO:15.

40. The kit according to claim 20, wherein said polynucleotide probe comprises at least 7 consecutive nucleotides of the sequence as set forth in SEQ ID NO:15.

41. The kit according to claim 21, wherein said polynucleotide probe comprises at least 7 consecutive nucleotides of the sequence as set forth in SEQ ID NO:15.
